(19) United States
(12) Patent Application Publication
(10) Pub. No.: US 2021/0330729 A1
(43) Pub. Date: Oct. 28, 2021

(54) DIETARY FIBER COMPOSITIONS

(71) Applicant: G&S Laboratories, Inc., Eagan, MN (US)

(72) Inventors: Robert A. Ganz, Eagan, MN (US); Mo E. Saremi, Eagan, MN (US); William Christopfel, Edina, MN (US)

(21) Appl. No.: 17/313,964

(22) Filed: May 6, 2021

Related U.S. Application Data
(63) Continuation-in-part of application No. 16/670,845, filed on Oct. 31, 2019.
(60) Provisional application No. 62/753,804, filed on Oct. 31, 2018.
(60) Provisional application No. 62/827,691, filed on Apr. 1, 2019.
(60) Provisional application No. 62/916,372, filed on Oct. 17, 2019.

(51) Int. Cl. A61K 36/68; A61K 31/732; A61K 31/7016; A61K 31/733; A61K 31/716; A61K 31/718; A23L 33/125; A23L 33/22; A23L 33/00; A23L 2/52

(52) U.S. Cl. CPC A61K 36/68; A61K 31/732; A61K 31/7016; A61K 31/733; A61K 31/716; A23V 2002/00; A23L 33/125; A23L 33/22; A23L 33/40; A23L 2/52; A61K 31/718

(57) ABSTRACT

The present disclosure relates to compositions comprising dietary fibers. For example, in some embodiments, the composition comprises two or more fibers (e.g., fibers such as wheat dextrin, inulin, pectin, or potato starch) each present in amount of about 2% to about 6% w/w of the composition. The present disclosure also relates to partially hydrolyzed dietary fibers and methods to prepare a partially hydrolyzed dietary. In some embodiments, the composition comprises further comprises an acid and/or a preservative present in an amount of about 0.01% to about 2% w/w of the composition. In some embodiments, the composition further includes a sweetener and/or a flavoring agent. Such compositions are useful in the treatment or improvement of gastrointestinal diseases.

### CROSS-REFERENCE TO RELATED APPLICATIONS

This application is a continuation-in-part of U.S. application Ser. No. 16/670,845, filed on Oct. 31, 2019, which claims priority to U.S. Provisional Application No. 62/916,372, filed on Oct. 17, 2019; U.S. Provisional Application No. 62/827,691, filed on Apr. 1, 2019; and U.S. Provisional Application No. 62/753,804, filed on Oct. 31, 2018; the contents of each of which are hereby incorporated by reference in their entireties.

### TECHNICAL FIELD

The present disclosure relates to compositions comprising dietary fibers. The present disclosure also relates to partially hydrolyzed dietary fibers, compositions comprising partially hydrolyzed dietary fibers, and methods to prepare a partially hydrolyzed dietary fiber. Such fibers are useful for treating or improving constipation, hemorrhoids, anal fissures, diarrhea, fecal incontinence, irritable bowel syndrome, inflammatory bowel disease (Crohn's disease and ulcerative colitis), microscopic colitis, leaky gut, certain metabolic syndromes such as glucose intolerance, prediabetes, diabetes, nonalcoholic fatty liver disease and obesity, and for altering the human microbiome in a favorable manner.

### BACKGROUND

Dietary fibers are a very important part of the human diet for a variety of reasons. For example, a diet high in insoluble dietary fiber (e.g., wheat bran) is recommended for individuals who require bulking of the stool, such as those with constipation, hemorrhoids, anal fissures, diarrhea, fecal incontinence and some types of irritable bowel syndrome, and a diet high in soluble fiber (e.g., psyllium, pectin, wheat dextrin, and oat products) can improve the glycogenic response to diet, which can improve blood glucose levels and some types of irritable bowel syndrome and can reduce the risk for type 2 diabetes, atherosclerosis, and stroke. Fiber intake may also be important with regard to systemic inflammation and/or leaky gut syndrome through, for example, the commensal relationship between the human gut and the bacteria within it. Bacteria in humans perform some functions that human cells cannot, such as generating some B vitamins, generating vitamin K, extracting nutrients from indigestible fiber, and generating short chain fatty acids (SCFAs). Importantly, SCFAs are derived from dietary fibers and are a source of energy for epithelial colonocytes. The types of SCFAs derived from the dietary fiber(s) depends on the dietary fiber, and the types of SCFAs have an impact on the health of colonocytes. An adequate amount of fiber is essential to maintain healthy gut ecology.

While the benefits of taking dietary fiber are well accepted, compliance can be poor with any fiber that is difficult to ingest, excessively course, granular or viscous. For example, psyllium (also known as ispaghula) is a specific soluble fiber that is commonly used as a supplement worldwide and can be used to fortify some breakfast cereals and baked goods. Psyllium is usually found in the form of husk, granules, capsules or powder, and it can also be used to fortify certain breakfast cereals and baked goods. It can also be used to treat irritable bowel syndrome, diarrhea and constipation, but also has FDA-approved claims for interfering with cholesterol absorption and improving glucose control in diabetes. Psyllium may also help prevent coronary artery disease. However, psyllium is difficult to take orally. It is generally packaged as a course powder that is unpalatable with a taste that is poorly liked by many. Moreover, the course, granular powder becomes thick and viscous in liquids, which makes it hard to physically ingest. Psyllium has to be mixed in a large volume of water, milk, juice or other beverages in order to dissolve, but because of its course granularity and highly viscous nature, it does not go completely into solution and remains a partially dissolved semisolid. It is has proven to be at best disagreeable to many adults, with many finding it revolting, and because of its nature, psyllium is impossible to take by the vast majority of children. Even many adults who take psyllium on a regular basis greatly dislike its flavor and texture and have to force it down. Psyllium granules can also be found in capsule form, but this form of the supplement is not absorbed as effectively and is very expensive. Currently psyllium in liquid form does not exist. Hence, there is a great need for a fiber preparation that has the same or similar fiber benefits and health effects as psyllium powder, and other coarse fibers, but tastes better, has a texture that is easier to take orally, and is readily able to be taken by children.

Accordingly, the present disclosure relates to compositions, e.g., liquid compositions, comprising dietary fibers (e.g., partially hydrolyzed dietary fibers). The present disclosure also relates to methods to prepare a partially hydrolyzed dietary fiber and using partially hydrolyzed dietary fiber(s) in liquid compositions.

### SUMMARY

Provided here are compositions comprising a first fiber and a second fiber.

In some embodiments, the first fiber is present in an amount of about 0.5% to about 5% w/w of the composition. In some embodiments, the first fiber is present in an amount of about 0.5% to about 2% w/w of the composition. In some embodiments, the first fiber is present in an amount of about 1% w/w of the composition. In some embodiments, the first fiber is present in an amount of about 2% to about 6% w/w of the composition. In some embodiments, the first fiber is present in an amount of about 4% w/w of the composition. In some embodiments, the first fiber is present in an amount of about 3% w/w of the composition.

In some embodiments, the first fiber is selected from the group consisting of: psyllium, pectin, guar, a lignin, cellulose, hemicellulose, resistant starch, a dextrin (e.g., a resistant dextrin), inulin, a chitin, pectin, a beta-glucan, potato starch, and an oligosaccharide. In some embodiments, the first fiber is pectin. In some embodiments, the first fiber is wheat dextrin. In some embodiments, the first fiber is inulin. In some embodiments, the first fiber is potato starch.

In some embodiments, the first fiber is partially hydrolyzed.

In some embodiments, the second fiber is present in an amount of about 0.5% to about 5% w/w of the composition. In some embodiments, the second fiber is present in an amount of about 0.5% to about 2% w/w of the composition. In some embodiments, the second fiber is present in an amount of about 1% w/w of the composition. In some embodiments, the second fiber is present in an amount of about 2% to about 6% w/w of the composition. In some embodiments, the second fiber is present in an amount of about 4% w/w of the composition. In some embodiments, the second fiber is present in an amount of about 3% w/w of the composition.

In some embodiments, the second fiber is selected from the group consisting of: psyllium, pectin, guar, a lignin, cellulose, hemicellulose, resistant starch, a resistant dextrin, inulin, a chitin, pectin, a beta-glucan, potato starch, and an oligosaccharide, wherein the second fiber is different from the first, third, and fourth fibers. In some embodiments, the second fiber is guar. In some embodiments, the second fiber is inulin. In some embodiments, the second fiber is potato starch. In some embodiments, the first fiber is wheat dextrin. In some embodiments, the second fiber is partially hydrolyzed.

In some embodiments, the ratio of the first fiber to the second fiber in the composition is about 1:4 to about 4:1 first fiber:second fiber. For example, about 1:3, about 2:5, about 1:2, about 2:3, about 1:1, about 2:1, about 5:2, about 3:1, or about 3:2 first fiber:second fiber.

In some embodiments, the composition further comprises a third fiber. In some embodiments, the third fiber is present in an amount of about 0.5% to about 5% w/w of the composition. In some embodiments, the third fiber is present in an amount of about 0.5% to about 2% w/w of the composition. In some embodiments, the third fiber is present in an amount of about 1% w/w of the composition. In some embodiments, the third fiber is present in an amount of about 2% to about 6% w/w of the composition. In some embodiments, the third fiber is present in an amount of about 4% w/w of the composition. In some embodiments, the third fiber is present in an amount of about 3% w/w of the composition.

In some embodiments, the third fiber is selected from the group consisting of: psyllium, pectin, guar, a lignin, cellulose, hemicellulose, resistant starch, a resistant dextrin, inulin, a chitin, pectin, a beta-glucan, potato starch, and an oligosaccharide, wherein the third fiber is different from the first, second, and fourth fibers. In some embodiments, the third fiber is inulin. In some embodiments, the second fiber is not inulin. In some embodiments, the third fiber is potato starch.

In some embodiments, the third fiber is partially hydrolyzed.

In some embodiments, the composition further comprises a fourth fiber. In some embodiments, the fourth fiber is present in an amount of about 0.5% to about 5% w/w of the composition. In some embodiments, the fourth fiber is present in an amount of about 0.5% to about 2% w/w of the composition. In some embodiments, the fourth fiber is present in an amount of about 1% w/w of the composition.

In some embodiments, the fourth fiber is selected from the group consisting of: psyllium, pectin, guar, a lignin, cellulose, hemicellulose, resistant starch, a resistant dextrin, inulin, a chitin, pectin, a beta-glucan, potato starch, and an oligosaccharide, wherein the fourth fiber is different from the first, second, and third fibers. In some embodiments, the fourth fiber is wheat dextrin.

In some embodiments, the fourth fiber is partially hydrolyzed.

In some embodiments, the composition further comprises a fifth fiber. In some embodiments, the fifth fiber is present in an amount of about 0.5% to about 5% w/w of the composition. In some embodiments, the fifth fiber is present in an amount of about 0.5% to about 2% w/w of the composition. In some embodiments, the fifth fiber is present in an amount of about 1% w/w of the composition.

In some embodiments, the fifth fiber is selected from the group consisting of:

psyllium, pectin, guar, a lignin, cellulose, hemicellulose, resistant starch, a resistant dextrin, inulin, a chitin, pectin, a beta-glucan, potato starch, and an oligosaccharide, wherein the fifth fiber is different from the first, second, third, and fourth fibers. In some embodiments, the fifth fiber is partially hydrolyzed.

In some embodiments, the composition further comprises an acid. In some embodiments, the composition further comprises a preservative. In some embodiments, the composition further comprises a sweetener. In some embodiments, the composition further comprises a flavoring agent.

In some embodiments, the composition further comprises a preservative and the preservative is present in an amount of about 0.01% to about 1% w/w of the composition. In some embodiments, the composition further comprises a sweetener and the sweetener is present in amount of about 0.001% to about 20% w/w of the composition.

In some embodiments, the composition further comprises a flavoring agent and the flavoring agent is present in amount of about 0.01% to about 2% w/w of the composition.

In some embodiments, the preservative is comprises: a palmitate, a benzoate, benzoic acid, a propionate, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, a tocopherol, or a combination thereof. In some embodiments, the benzoate is sodium benzoate. In some embodiments, the sorbate is selected from the group consisting of: calcium sorbate, potassium sorbate, sodium sorbate, and a combination of two or more thereof.

In some embodiments, the preservative comprises sodium benzoate and potassium sorbate. In some embodiments, the preservative comprises sodium sorbate and sodium benzoate and the sodium sorbate and sodium benzoate together are present in an amount of about 0.1% to about 0.3% w/w of the composition. In some embodiments, the preservative comprises sodium sorbate and sodium benzoate, and the sodium sorbate is present in an amount of about 0.05% to about 0.2% w/w of the composition and the sodium benzoate is present in an amount of about 0.05% to about 0.2% w/w of the composition.

In some embodiments, the composition further comprises a sweetener and the sweetener comprises: an intense sweetener, a natural sugar, or a combination thereof. In some embodiments, the composition further comprises a sweetener and the sweetener comprises: sorbitol, sucrose, a saccharin, a cyclamate, aspartame, sucralose, thaumatin, acesulfam K, xylose, ribose, glucose, mannose, galactose, fructose, dextrose, sucrose, maltose, starch or corn syrup, sorbitol, xylitol, mannitol, glycerin, or a combination thereof.

In some embodiments, the composition further comprises a flavoring agent and the flavoring agent comprises: a natural flavoring agent, a natural fruit flavoring agent, an artificial flavoring agent, an artificial fruit flavoring agent, a flavor enhancer, or a combination thereof.

Also provided herein are compositions comprising wheat dextrin and inulin. In some embodiments, the composition comprises: wheat dextrin present in an amount of about 2% to about 6% w/w of the composition; and inulin present in an amount of about 2% to about 6% w/w of the composition. In some embodiments, the composition comprises wheat dextrin present in an amount of about 4% w/w of the composition; and inulin present in an amount of about 4% w/w of the composition. In some embodiments, the composition comprises wheat dextrin present in an amount of about 3% w/w of the composition; and inulin present in an amount of about 3% w/w of the composition.

In some embodiments, the ratio of the wheat dextrin to the inulin in the composition is about 1:4 to about 4:1 wheat dextrin:inulin. For example, about 1:3, about 2:5, about 1:2, about 2:3, about 1:1, about 2:1, about 5:2, about 3:1, or about 3:2 wheat dextrin:inulin.

Also provided herein are compositions comprising pectin and inulin. In some embodiments, the composition comprises pectin present in an amount of about 2% to about 6% w/w of the composition; and inulin present in an amount of about 2% to about 6% w/w of the composition. In some embodiments, the composition comprises pectin present in an amount of about 4% w/w of the composition; and inulin present in an amount of about 4% w/w of the composition. In some embodiments, the composition comprises pectin present in an amount of about 3% w/w of the composition; and inulin present in an amount of about 3% w/w of the composition.

In some embodiments, the ratio of the pectin to the inulin in the composition is about 1:4 to about 4:1 pectin:inulin. For example, about 1:3, about 2:5, about 1:2, about 2:3, about 1:1, about 2:1, about 5:2, about 3:1, or about 3:2 pectin:inulin.

Also provided herein are compositions comprising pectin and potato starch. In some embodiments, the composition comprises pectin present in an amount of about 2% to about 6% w/w of the composition; and potato starch present in an amount of about 2% to about 6% w/w of the composition. In some embodiments, the composition comprises pectin present in an amount of about 4% w/w of the composition; and potato starch present in an amount of about 4% w/w of the composition. In some embodiments, the composition comprises pectin present in an amount of about 3% w/w of the composition; and potato starch present in an amount of about 3% w/w of the composition.

In some embodiments, the ratio of the pectin to the potato starch in the composition is about 1:4 to about 4:1 pectin:potato starch. For example, about 1:3, about 2:5, about 1:2, about 2:3, about 1:1, about 2:1, about 5:2, about 3:1, or about 3:2 pectin:potato starch.

Also provided herein are compositions comprising potato starch and inulin. In some embodiments, the composition comprises potato starch present in an amount of about 2% to about 6% w/w of the composition and inulin present in an amount of about 2% to about 6% w/w of the composition. In some embodiments, the composition comprises potato starch present in an amount of about 4% w/w of the composition; and inulin present in an amount of about 4% w/w of the composition. In some embodiments, the composition comprises potato starch present in an amount of about 3% w/w of the composition; and inulin present in an amount of about 4% w/w of the composition. In some embodiments, the composition comprises potato starch present in an amount of about 3% w/w of the composition; and inulin present in an amount of about 3% w/w of the composition.

In some embodiments, the ratio of the potato starch to the inulin in the composition is about 1:4 to about 4:1 potato starch:inulin. For example, about 1:3, about 2:5, about 1:2, about 2:3, about 1:1, about 2:1, about 5:2, about 3:1, or about 3:2 potato starch:inulin.

Also provided herein are compositions comprising wheat dextrin and potato starch. In some embodiments, the composition comprises wheat dextrin present in an amount of about 2% to about 6% w/w of the composition and potato starch present in an amount of about 2% to about 6% w/w of the composition. In some embodiments, the composition comprises wheat dextrin present in an amount of about 4% w/w of the composition; and potato starch present in an amount of about 4% w/w of the composition. In some embodiments, the composition comprises wheat dextrin present in an amount of about 3% w/w of the composition; and potato starch present in an amount of about 4% w/w of the composition. In some embodiments, the composition comprises wheat dextrin present in an amount of about 3% w/w of the composition; and potato starch present in an amount of about 3% w/w of the composition.

In some embodiments, the ratio of the wheat dextrin to the potato starch in the composition is about 1:4 to about 4:1 wheat dextrin:potato starch. For example, about 1:3, about 2:5, about 1:2, about 2:3, about 1:1, about 2:1, about 5:2, about 3:1, or about 3:2 wheat dextrin:potato starch.

Also provided herein are compositions comprising wheat dextrin and pectin. In some embodiments, the composition comprises pectin present in an amount of about 2% to about 6% w/w of the composition and wheat dextrin present in an amount of about 2% to about 6% w/w of the composition. In some embodiments, the composition comprises pectin present in an amount of about 4% w/w of the composition; and wheat dextrin present in an amount of about 4% w/w of the composition. In some embodiments, the composition comprises pectin present in an amount of about 3% w/w of the composition; and wheat dextrin present in an amount of about 4% w/w of the composition. In some embodiments, the composition comprises pectin present in an amount of about 3% w/w of the composition; and wheat dextrin present in an amount of about 3% w/w of the composition.

In some embodiments, the ratio of the pectin to the wheat dextrin in the composition is about 1:4 to about 4:1 pectin:wheat dextrin. For example, about 1:3, about 2:5, about 1:2, about 2:3, about 1:1, about 2:1, about 5:2, about 3:1, or about 3:2 pectin:wheat dextrin.

Also provided herein are compositions comprising one or more partially hydrolyzed fibers. In some embodiments, the one or more partially hydrolyzed fibers are present in an amount of about 0.5% to about 10% w/w of the composition. In some embodiments, the one or more partially hydrolyzed fibers are present in an amount of about 0.5% to about 5% w/w of the composition. In some embodiments, the one or more partially hydrolyzed fibers are present in an amount of about 0.5% to about 3% w/w of the composition. In some embodiments, the one or more partially hydrolyzed fibers are present in an amount of about 2% w/w of the composition. In some embodiments, the one or more partially hydrolyzed fibers are present in an amount of about 1% w/w of the composition. In some embodiments, the one or more partially hydrolyzed fibers are present in an amount of about 2% to about 6% w/w of the composition. In some embodiments, the one or more partially hydrolyzed fibers are present in an amount of about 3% to about 5% w/w of the composition. In some embodiments, the one or more partially hydrolyzed fibers are present in an amount of about 4% w/w of the composition. In some embodiments, the one or more partially hydrolyzed fibers are present in an amount of about 4% to about 8% w/w of the composition. In some embodiments, the one or more partially hydrolyzed fibers are present in an amount of about 5% to about 7% w/w of the composition. In some embodiments, the one or more partially hydrolyzed fibers are present in an amount of about 6% w/w of the composition.

In some embodiments, the one or more partially hydrolyzed fibers are selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, a partially hydrolyzed oligosaccharide, and partially hydrolyzed potato starch. In some embodiments, the one or more partially hydrolyzed fibers is partially hydrolyzed psyllium. In some embodiments, the one or more partially hydrolyzed fibers is partially hydrolyzed pectin.

Also provided herein are compositions comprising a first partially hydrolyzed fiber and a second partially hydrolyzed fiber. In some embodiments, the first partially hydrolyzed fiber is present in an amount of about 0.5% to about 8% w/w of the composition. In some embodiments, the first partially hydrolyzed fiber is present in an amount of about 2% to about 6% w/w of the composition. In some embodiments, the first partially hydrolyzed fiber is present in an amount of about 4% w/w of the composition. In some embodiments, the first partially hydrolyzed fiber is present in an amount of about 0.5% to about 2% w/w of the composition. In some embodiments, the first partially hydrolyzed fiber is present in an amount of about 1% w/w of the composition.

In some embodiments, the first partially hydrolyzed fiber is selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, partially hydrolyzed potato starch, and a partially hydrolyzed oligosaccharide. In some embodiments, the first partially hydrolyzed fiber is partially hydrolyzed pectin.

In some embodiments, the second partially hydrolyzed fiber is present in an amount of about 0.5% to about 8% w/w of the composition. In some embodiments, the second partially hydrolyzed fiber is present in an amount of about 2% to about 6% w/w of the composition. In some embodiments, the second partially hydrolyzed fiber is present in an amount of about 4% w/w of the composition. In some embodiments, the second partially hydrolyzed fiber is present in an amount of about 0.5% to about 2% w/w of the composition. In some embodiments, the second partially hydrolyzed fiber is present in an amount of about 1% w/w of the composition.

In some embodiments, the second partially hydrolyzed fiber is present in an amount of about 1% w/w of the composition. In some embodiments, the second partially hydrolyzed fiber is selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, partially hydrolyzed potato starch, and a partially hydrolyzed oligosaccharide, wherein the second partially hydrolyzed fiber is different from the first, third, and fourth partially hydrolyzed fibers. In some embodiments, the second partially hydrolyzed fiber is partially hydrolyzed guar. In some embodiments, the second partially hydrolyzed fiber is partially hydrolyzed inulin.

In some embodiments, the composition further comprises a third partially hydrolyzed fiber. In some embodiments, the third partially hydrolyzed fiber is present in an amount of about 0.5% to about 8% w/w of the composition. In some embodiments, the third partially hydrolyzed fiber is present in an amount of about 2% to about 6% w/w of the composition. In some embodiments, the third partially hydrolyzed fiber is present in an amount of about 4% w/w of the composition. In some embodiments, the third partially hydrolyzed fiber is present in an amount of about 0.5% to about 2% w/w of the composition. In some embodiments, the third partially hydrolyzed fiber is present in an amount of about 1% w/w of the composition. In some embodiments, the third partially hydrolyzed fiber is selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, partially hydrolyzed potato starch, and a partially hydrolyzed oligosaccharide, wherein the third partially hydrolyzed fiber is different from the first, second, and fourth partially hydrolyzed fibers. In some embodiments, the third partially hydrolyzed fiber is partially hydrolyzed inulin.

In some embodiments, the composition further comprises a fourth partially hydrolyzed fiber. In some embodiments, the fourth partially hydrolyzed fiber is present in an amount of about 0.5% to about 8% w/w of the composition. In some embodiments, the fourth partially hydrolyzed fiber is present in an amount of about 2% to about 6% w/w of the composition. In some embodiments, the fourth partially hydrolyzed fiber is present in an amount of about 4% w/w of the composition. In some embodiments, the fourth partially hydrolyzed fiber is present in an amount of about 0.5% to about 2% w/w of the composition. In some embodiments, the fourth partially hydrolyzed fiber is present in an amount of about 1% w/w of the composition.

In some embodiments, the fourth partially hydrolyzed fiber is selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, partially hydrolyzed potato starch, and a partially hydrolyzed oligosaccharide, wherein the fourth partially hydrolyzed fiber is different from the first, second, and third partially hydrolyzed fibers. In some embodiments, the fourth partially hydrolyzed fiber is partially hydrolyzed wheat dextrin.

In some embodiments, the composition further comprises a fifth partially hydrolyzed fiber. In some embodiments, the fifth partially hydrolyzed fiber is present in an amount of about 0.5% to about 8% w/w of the composition. In some embodiments, the fifth partially hydrolyzed fiber is present in an amount of about 2% to about 6% w/w of the composition. In some embodiments, the fifth partially hydrolyzed fiber is present in an amount of about 4% w/w of the composition. In some embodiments, the fifth partially hydrolyzed fiber is present in an amount of about 0.5% to about 2% w/w of the composition. In some embodiments, the fifth partially hydrolyzed fiber is present in an amount of about 1% w/w of the composition.

In some embodiments, the fifth partially hydrolyzed fiber is selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, partially hydrolyzed potato starch, and a partially hydrolyzed oligosaccharide, wherein the fifth partially hydrolyzed fiber is different from the first, second, third, and fourth partially hydrolyzed fibers.

In some embodiments, the composition further comprises an acid. In some embodiments, the composition further comprises a preservative. In some embodiments, the composition further comprises a sweetener. In some embodiments, the composition further comprises a flavoring agent.

Also provided herein are compositions comprising: a partially hydrolyzed psyllium present in amount of about 0.5% to about 10% w/w of the composition; an acid; and a preservative present in an amount of about 0.01% to about 2% w/w of the composition. In some embodiments, the preservative is a combination of sodium sorbate and sodium benzoate. In some embodiments, the hydrolyzed psyllium is present in amount of about 2% w/w of the composition; and the sodium sorbate and sodium benzoate together are present in an amount of about 0.2% w/w of the composition.

Also provided herein are compositions comprising: a partially hydrolyzed pectin present in an amount of about 0.5% to about 10% w/w of the composition; an acid; and a preservative present in an amount of about 0.01% to about 2% w/w of the composition. In some embodiments, the preservative is a combination of sodium sorbate and sodium benzoate. In some embodiments, the hydrolyzed pectin is present in an amount of about 6% w/w of the composition; and the sodium sorbate and sodium benzoate together are present in an amount of about 0.2% w/w of the composition.

Also provided herein are compositions comprising: a partially hydrolyzed pectin; and a partially hydrolyzed inulin. Also provided herein are compositions comprising: a partially hydrolyzed pectin present in an amount of about 2% to about 6% w/w of the composition; and a partially hydrolyzed inulin present in an amount of about 2% to about 6% w/w of the composition. Also provided herein are compositions comprising: a partially hydrolyzed pectin present in an amount of about 4% w/w of the composition; and a partially hydrolyzed inulin present in an amount of about 4% w/w of the composition.

Also provided herein are compositions comprising: a partially hydrolyzed pectin; a partially hydrolyzed guar; a partially hydrolyzed inulin; and a partially hydrolyzed wheat dextrin. Also provided herein are compositions comprising: a partially hydrolyzed pectin present in an amount of about 0.5% to about 2% w/w of the composition; a partially hydrolyzed guar present in an amount of about 0.5% to about 2% w/w of the composition; a partially hydrolyzed inulin present in an amount of about 0.5% to about 2% w/w of the composition; and a partially hydrolyzed wheat dextrin present in an amount of about 0.5% to about 2% w/w of the composition.

Also provided herein are compositions comprising: a partially hydrolyzed pectin present in an amount of about 1% w/w of the composition; a partially hydrolyzed guar present in an amount of about 1% w/w of the composition; a partially hydrolyzed inulin present in an amount of about 1% w/w of the composition; and a partially hydrolyzed wheat dextrin present in an amount of about 1% w/w of the composition.

In some embodiments, a composition as described herein further comprises an acid. In some embodiments, the acid has a molarity of about 0.01 to about 0.1 M. In some embodiments, the acid has a molarity of about 0.05 M. In some embodiments, the acid has a molarity of about 0.013 M. In some embodiments, the acid is selected from the group consisting of: HCl, HI, HBr, HClO₄, HClO₃, HNO₃, H₂SO₄, phosphoric acid, phosphorous acid, acetic acid, oxalic acid, ascorbic acid, carbonic acid, sulfurous acid, tartaric acid, citric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof. In some embodiments, the acid is a strong acid. In some embodiments, the strong acid is selected from the group consisting of: HCl, HI, HBr, HClO₄, HClO₃, HNO₃, H₂SO₄, and a combination thereof. In some embodiments, the acid is a weak acid. In some embodiments, the weak acid is selected from the group consisting of: phosphorous acid, acetic acid, oxalic acid, phosphoric acid, ascorbic acid, carbonic acid, citric acid, sulfurous acid, tartaric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof. In some embodiments, the weak acid is phosphoric acid. In some embodiments, the weak acid is citric acid.

In some embodiments, the composition has a pH of about 3 to about 5. In some embodiments, the composition has a pH of about 3.5 to about 4. In some embodiments, the composition has a pH of about 3.8. In some embodiments, the composition has a pH of about 3.8 to about 4.2.

In some embodiments, the remaining part of the composition is water or buffered water. In some embodiments, the composition is a liquid composition. In some embodiments, the liquid composition is selected from the group consisting of: a solution, a syrup, a suspension, and a concentrate.

Also provided herein are methods for reducing inflammation in a subject in need thereof comprising administering to the subject a composition as described herein. In some embodiments, provided herein are methods for improving intestinal health in a subject in need thereof comprising administering to the subject a composition as described herein. In some embodiments, provided herein are methods for improving a gastrointestinal disease in a subject in need thereof comprising administering to the subject a composition as described herein.

Also provided herein are methods for improving diabetes or prediabetes in a subject in need thereof comprising administering to the subject a composition as described herein. In some embodiments, provided herein are methods for improving blood glucose levels in a subject in need thereof comprising administering to the subject a composition as described herein. In some embodiments, the subject is administered a composition comprising: wheat dextrin present in an amount of about 4% w/w of the composition; and potato starch present in an amount of about 4% w/w of the composition.

Also provided herein are methods for preparing a partially hydrolyzed fiber, the method comprising contacting one or more fibers with an effective amount of a hydrolyzing agent. In some embodiments, the method comprises contacting one or more fibers with an effective amount of a hydrolyzing agent to form a mixture and incubating the mixture for a period of time to form an incubated mixture. In some embodiments, the period of time is about 5 to about 8 hours. In some embodiments, the period of time is about 6 to about 7 hours. In some embodiments, the mixture is at about room temperature during the period of time.

In some embodiments, the method further comprises contacting one or more fibers with an effective amount of a hydrolyzing agent to form a mixture and heating the mixture to a temperature of about 50° C. to about 85° C. to form a heated mixture. In some embodiments, the method comprises contacting one or more fibers with an effective amount of a hydrolyzing agent to form a mixture and heating the mixture to a temperature of about 60° C. to about 75° C. to form a heated mixture. In some embodiments, the method comprises contacting one or more fibers with an effective amount of a hydrolyzing agent to form a mixture and heating the mixture to a temperature of about 65° C. to about 70° C. to form a heated mixture. In some embodiments, the method comprises contacting one or more fibers with an effective amount of a hydrolyzing agent to form a mixture; heating the mixture to about 65° C. to about 70° C.; and incubating the heated mixture for a period of time of about 6.5 hours at a temperature of about 65° C. to about 70° C. to form a heated and incubated mixture.

In some embodiments, the method further comprises cooling the heated mixture to form a partially hydrolyzed fiber mixture. In some embodiments, the method further comprises cooling the heated mixture rapidly to form a partially hydrolyzed fiber mixture. In some embodiments, the method further comprises cooling the heated mixture with mixing to form a partially hydrolyzed fiber mixture. In some embodiments, the method comprises cooling the heated mixture to about room temperature. In some embodiments, the method further comprises cooling the heated and incubated mixture to form a partially hydrolyzed fiber mixture. In some embodiments, the method further comprises cooling the heated and incubated mixture rapidly to form a partially hydrolyzed fiber mixture. In some embodiments, the method further comprises cooling the heated and incubated mixture with mixing to form a partially hydrolyzed fiber mixture. In some embodiments, the method comprises cooling the heated and incubated mixture to about room temperature.

Also provided herein are methods for preparing a partially hydrolyzed fiber, the method comprising: contacting one or more fibers with an effective amount of a hydrolyzing agent to form a mixture; heating the mixture to about 50° C. to about 80° C.; holding the heated mixture at about 50° C. to about 80° C. for a period of time; and cooling the heated mixture to form a partially hydrolyzed fiber mixture.

In some embodiments, the mixture is heated to about 60° C. to about 75° C. In some embodiments, the mixture is heated to about 65° C. to about 70° C. In some embodiments, the heated mixture is held at a temperature of about 60° C. to about 75° C. In some embodiments, the heated mixture is held at a temperature of about 65° C. to about 70° C. In some embodiments, the mixture is heated rapidly. In some embodiments, the method further comprises stirring the mixture during heating. In some embodiments, the method further comprises stirring the heated mixture. In some embodiments, the heated mixture is cooled rapidly. In some embodiments, the method further comprises stirring the mixture during cooling. In some embodiments, the mixture is cooled to about room temperature.

Also provided herein are methods for preparing a partially hydrolyzed fiber, the method comprising: contacting one or more fibers with an effective amount of a hydrolyzing agent to form a mixture; and incubating the mixture at about room temperature for a period of time to form a partially hydrolyzed fiber mixture.

In some of any of the above methods, the method further comprises adding one or more preservatives to the partially hydrolyzed fiber mixture. In some embodiments, the partially hydrolyzed fiber mixture is filtered. In some embodiments, the pH of the partially hydrolyzed fiber mixture is brought to about 3 to about 5. In some embodiments, the pH of the partially hydrolyzed fiber mixture is brought to about 3.8. In some embodiments, the method further comprises adding one or more sweeteners. In some embodiments, the method further comprises adding one or more flavoring agents.

In some embodiments, the selected hydrolyzing agent is selected from the group consisting of: a hydrolytic enzyme and an acid. In some embodiments, the hydrolyzing agent is an acid. In some embodiments, the acid is selected from the group consisting of: HCl, HI, HBr, HClO₄, HClO₃, HNO₃, H₂SO₄, phosphoric acid, phosphorous acid, acetic acid, oxalic acid, ascorbic acid, carbonic acid, sulfurous acid, tartaric acid, citric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof. In some embodiments, the acid is a strong acid. In some embodiments, the strong acid is selected from the group consisting of: HCl, HI, HBr, HClO₄, HClO₃, HNO₃, H₂SO₄, and a combination thereof. In some embodiments, the acid is a weak acid. In some embodiments, the weak acid is selected from the group consisting of: phosphorous acid, acetic acid, oxalic acid, phosphoric acid, ascorbic acid, carbonic acid, citric acid, sulfurous acid, tartaric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof. In some embodiments, the weak acid is phosphoric acid.

In some embodiments, the fiber is selected from the group consisting of: psyllium, pectin, guar, a lignin, cellulose, hemicellulose, resistant starch, a resistant dextrin, inulin, a chitin, pectin, a beta-glucan, potato starch, an oligosaccharide, and a combination thereof. In some embodiments, the fiber is psyllium. In some embodiments, the fiber is pectin.

In some embodiments, the one or more fibers are present in an amount of about 0.5% to about 10% w/w of the mixture. In some embodiments, the one or more fibers are present in an amount of about 0.5% to about 5% w/w of the composition. In some embodiments, the one or more fibers are present in an amount of about 0.5% to about 3% w/w of the composition. In some embodiments, the one or more fibers are present present in an amount of about 2% w/w of the composition. In some embodiments, the one or more fibers are present in an amount of about 1% w/w of the composition. In some embodiments, the one or more fibers are present in an amount of about 2% to about 6% w/w of the composition. In some embodiments, the one or more fibers are present in an amount of about 3% to about 5% w/w of the composition. In some embodiments, the one or more fibers are present in an amount of about 4% w/w of the composition. In some embodiments, the one or more fibers are present in an amount of about 4% to about 8% w/w of the composition. In some embodiments, the one or more fibers are present in an amount of about 5% to about 7% w/w of the composition. In some embodiments, the one or more fibers are present in an amount of about 6% w/w of the composition.

Also provided herein is a partially hydrolyzed fiber. In some embodiments, the partially hydrolyzed fiber is a partially hydrolyzed soluble fiber. In some embodiments, the partially hydrolyzed soluble fiber is selected from the group consisting of: partially hydrolyzed psyllium, partially hydrolyzed pectin, partially hydrolyzed wheat dextrin, a partially hydrolyzed beta-glucan product, and a combination thereof. In some embodiments, the partially hydrolyzed fiber is selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, partially hydrolyzed potato starch, a partially hydrolyzed oligosaccharide, and a combination thereof. In some embodiments, the partially hydrolyzed fiber is partially hydrolyzed psyllium. In some embodiments, the partially hydrolyzed fiber is partially hydrolyzed pectin.

The details of one or more embodiments of the invention are set forth in the accompanying drawings and the description below. Other features, objects, and advantages of the invention will be apparent from the description and drawings, and from the claims.

### DETAILED DESCRIPTION

Dietary fibers (also referred to herein as “fibers”) include non-starch polysaccharides and other plant components such as cellulose, hemicellulose, resistant starch, resistant dextrins, inulin, lignins, chitins, pectins, guar, beta-glucans, and oligosaccharides. Insoluble dietary fibers can help bulk stool, which is recommended for individuals with constipation, hemorrhoids, anal fissures, diarrhea, fecal incontinence and some types of irritable bowel syndrome, and soluble, or partially soluble, dietary fibers, such as psyllium, pectin, wheat dextrin, and beta-glucan (e.g., from oat products), can improve the glycogenic response to diet. Additionally, fiber intake plays an important role regarding the relationship between the human gut and the bacteria within it. Humanoids have co-evolved with bacteria for over 1 million years, and humans have co-evolved with bacteria for about the last 300,000 years. It is estimated that humans contain over 1 trillion bacterial cells, mostly on the skin and in the gut. Of the bacteria found in the gut, most are in the colon. To keep the bacteria from causing inflammation in the colon wall, humans have evolved 4 separate defenses: 1) the bacterial ecology is organized such that humans are mainly populated by non-inflammatory species that keep pathogens (e.g., Clostridium difficile) and pro-inflammatory commensals in check; 2) humans have a thick mucus layer that contains viral phages that destroy bacteria; 3) below the mucus layer is the epithelium with tight junctions; and 4) below that is the submucosa with plasma cells that secrete IgA, lymphocytes, antimicrobial peptides (AMPs), etc. Some bacteria have co-evolved with humans as commensals, for example, bacteria in humans perform some functions that human cells cannot, such as generating some B vitamins, generating vitamin K, extracting nutrients from indigestible fiber, and generating short chain fatty acids (SCFAs). Certain SFCAs such as proprionic acid, butyric acid and acetic acid are necessary for colon health, because they are the sole nutritional source for colonocytes; without SCFAs, the lining of the colon would atrophy and potentially lead to the development of colitis. There is a very important interplay between SCFAs, gut microbiota, and host energy metabolism, for example, soluble fiber typically becomes viscous in the human gastrointestinal tract and certain types of fiber can drive the growth of certain gut bacterial species. While some fibers, for example, psyllium, are not particularly fermentable and so, they do not generate many SCFAs, hydrolysis of psyllium generates shorter chain oligosaccharides, e.g., various monosaccharides, di saccharides, trisaccharides, etc. leading to carbohydrate monomers and polymers of variable length, which are more fermentable.

Humans are largely populated by four bacterial genuses: bacteroides, firmacutes, proteobacters, and acintobacters. While developed and undeveloped human populations have very different distributions of these genuses and species, across all human populations there are consistently 15-17 key foundational species within these groups that sustain a healthy gut ecology. However, if these foundational species are suppressed (e.g., by antibiotics or a diet high in fat and sugar and low in fiber), other commensals may grow more populous, and the gut can lose foundational species. The other new commensals may have a more inflammatory interaction with colonocytes, may not generate the same amount of SCFAs, and/or may generate the wrong type of SCFAs. This may lead to an inflammatory interaction or atrophy of colonocytes, which may open up the tight cell junctions potentially causing a leaky gut, thin out or change the mucus lining, or cause an increase in submucosal white cell populations.

As such, dietary fiber intake may also have a role in systemic inflammation and leaky gut syndrome, e.g., a breach in the mucosal barrier of the intestine that may allow bacteria, bacterial antigens, and bacterial lipopolysaccharide (LPS) to enter the blood stream, which may cause generalized or local inflammation and possibly autoimmune diseases. For example, if the bacteria/LPS products get into the liver, in the presence of obesity, humans can get non-alcoholic fatty liver disease or nonalcoholic steatohepatitis (NAFLD/NASH) and cirrhosis; in the pancreas and periphery, humans can get insulin resistance; in the colon, humans can get irritable bowel syndrome; and in the coronary arteries we can get atherosclerosis. Other diseases that may be linked to leaky gut include CAD/MI, clotting disorders, and celiac sprue and inflammatory bowel diseases (e.g., Crohn's and ulcerative colitis), which are consistently associated with an increased risk in many autoimmune diseases. Hence, dietary fibers, and the types of fibers ingested, play an important, multifaceted and complex role in human health and disease.

### Definitions

As used herein, the phrases an “effective amount” or a “therapeutically effective amount” of an active agent or ingredient, or pharmaceutically active agent or ingredient, refer to an amount of the active agent or pharmaceutically active agent sufficient enough to reduce or eliminate one or more symptoms of the disorder or to effect a cure upon administration. Effective amounts of the active agent or pharmaceutically active agent will vary with the kind of active agent or pharmaceutically active agent chosen, the particular condition or conditions being improved or treated, the severity of the condition, the duration of the treatment, the specific components of the composition being used, and like factors.

As used herein, “subject” or “patient” refers to any subject, particularly a mammalian subject, for whom diagnosis, prognosis, or therapy is desired, for example, a human.

As used herein, a “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or provide improvement to a patient or subject's quality of life.

As used herein “improvement” of or “improving” a disease, disorder, or condition means reducing the severity of a disease, disorder, or condition, reducing the severity of one or more symptoms associated therewith, or increasing a patient or subject's quality of life

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 to and including 22.

As used herein, “fibers” are carbohydrates mostly derived from plants that are resistant to digestion by human enzymes. In some embodiments, a fiber is a polysaccharide.

As used herein, “partially hydrolyzed fibers” or “a partially hydrolyzed fiber” refers to fibers that have hydrolysable chemical bonds that have been hydrolyzed, but where not all of the hydrolyzable chemical bonds have been hydrolyzed. For example, wherein from about 1% to about 99% of the hydrolysable chemical bonds have been hydrolyzed. For example, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the hydrolysable chemical bonds have been hydrolyzed. In some embodiments, about 10% to about 99% of the hydrolysable chemical bonds have been hydrolyzed. The extent of hydrolysis of a polysaccharide (e.g., a fiber) can be determined by any of the methods known to one of skill in the art. For example, the number of reducing ends of polysaccharides (e.g., a fibers) can be determined and that can be used to calculate the number of sugars per chain to determine the length of the polysaccharide (e.g., the fiber). In some embodiments, these methods can be colorimetric and/or based on the ability of aldehydes to reduce other molecules. As another example, the molecular weight of a polymer, such as a polysaccharide, fiber, or partially hydrolyzed fiber, can be determined by a variety of techniques. Non-limiting examples of techniques to determine the molecular weight of a polymer include: gel permeation chromatography (GPC), static light scattering (SLS), and intrinsic viscosity (IV). In some cases, certain types of fibers can partially hydrolyze when added to water.

The chain length of a partially hydrolyzed polysaccharide (e.g., a partially hydrolyzed fiber) can be related to the viscosity of the composition comprising the partially hydrolyzed polysaccharide (e.g., a partially hydrolyzed fiber). In some embodiments, the extent of hydrolysis of the partially hydrolyzed fiber is quantified by the viscosity of the composition comprising the partially hydrolyzed fiber. The viscosity of a solution comprising one or more fibers can depend on the extent of the hydrolysis of the one or more fibers. For example, the viscosity of a solution comprising one or more partially hydrolyzed fibers can decrease with increasing hydrolysis of one or more of the fibers. In some embodiments, the greater the extent of hydrolysis of the partially hydrolyzed fiber (e.g., the higher the percentage of hydrolyzed chemical bonds), the greater the decrease in the viscosity of a solution comprising the partially hydrolyzed fiber compared to a solution comprising the unhydrolyzed fiber.

“Room temperature” or “RT” refers to the ambient temperature of a typical laboratory, which is typically around 25° C.

The term “dietary fiber combination” as used herein means a product that results from the mixing or combining of more than one dietary fiber and includes both fixed and non-fixed combinations of the dietary fibers. The term “fixed combination” means that the dietary fibers, e.g. a first fiber and a second fiber, are both administered to a subject simultaneously in the form of a single entity or dosage. The term “non-fixed combination” means that the dietary fibers, e.g. a first fiber and a second fiber, are both administered to a patient as separate entities either simultaneously, concurrently or sequentially with no specific time limits, wherein such administration provides therapeutically effective levels of the 2 dietary fibers in the body of the patient. The latter also applies to the administration of 3 or more dietary fibers.

### Dietary Fiber Compositions

Provided herein are compositions comprising two or more fibers (e.g., a first fiber, a second fiber, a third fiber, a fourth fiber, and a fifth fiber). Non-limiting examples of a fiber include non-starch polysaccharides and other plant components such as cellulose, hemicellulose, resistant starch, resistant dextrins, inulin, lignins, chitins, pectins, guar, beta-glucans, potato starch and oligosaccharides. Fiber is mostly derived from plants, and it consists of the edible portion of a plant-based diet that cannot be digested by the human digestive system. Fiber is typically divided into soluble fiber, i.e., fiber that can dissolve in water, and insoluble fiber, which cannot dissolve in water. Insoluble fiber comes from the cell walls of plants and this type of fiber does not dissolve in water. Examples of foods that contain insoluble fiber include potato starch, wheat, rye and other grains such as bran. Some types of insoluble fibers contain complex carbohydrates such as resistant starches and can be fermented by bacteria. Thus, some types of insoluble fiber can also act as a prebiotic and produce short chain fatty acids (SCFAs), which are physiologically active, but other types of insoluble fibers, such as lignins, cannot be fermented, and they largely act as bulking agents. Soluble fiber is composed of carbohydrates, and certain types can be readily fermented by gut bacteria in the small bowel or colon. Non-limiting examples of foods that contain soluble fiber include fruits, oats, barley, and legumes such as peas and corn. Non-limiting examples of soluble fibers include psyllium, pectin, wheat dextrin, oligosaccharides, and beta-glucan (e.g., from oat products). Oligosaccharides can include fructooligosaccharides (FOS) also known as oligofructose, maltose, iso-maltose, maltosyl-iso-malto-oligosaccharides, galactooligosaccharides, and mannan-oligosaccharides (MOS). FOS are composed of linear chains of fructose units that are linked by beta (2-1) bonds. The number of fructose units ranges from 2 to 60 and often terminate in a glucose unit. Inulin and FOS can be derived from chicory root fiber. In some embodiments, FOS is degraded (e.g., hydrolyzed) inulin. In some embodiments, FOS is synthesized enzymatically (see, e.g., Lorenzoni et al. Carbohydr. Polym. 2014; 103:193-7). Commercial psyllium is composed mainly of arabinose (22%), xylose (57%), and uronic acids (10-15%), with small amounts of galactose, rhamnose, glucose, and mannose; it is a highly branched acidic arabinoxylan with a high molecular weight. Psyllium is derived from the seeds of the plant genus Plantago, including Plantago ovata, an herb mainly grown in India.

In some embodiments, the composition further comprises one or more of an acid, a preservative, a sweetener, and a flavoring agent.

In some embodiments, the remaining part of the composition is water or buffered water. For example, water or buffered water can be added to account for the rest of the composition such that the total amount of the composition is 100% w/w (e.g., if the composition comprises a first fiber present at 4% w/w of the composition, water can account for the remaining 96% w/w of the composition). Accordingly, in some embodiments, a composition as provided herein can be a liquid. For example, the composition may be an aqueous solution comprising two or more fibers. In some embodiments, the liquid composition is selected from the group consisting of: a solution, a syrup, a suspension, and a concentrate.

In some embodiments, the composition has two fibers (e.g., a first fiber and a second fiber). In some embodiments, the composition has three fibers (e.g., at least a first fiber, a second fiber, and a third fiber). In some embodiments, the composition has four fibers (e.g., a first fiber, a second fiber, a third fiber, and a fourth fiber). In some embodiments, the composition has five fibers (e.g., a first fiber, a second fiber, a third fiber, a fourth fiber, and a fifth fiber).

In some embodiments, the two or more fibers (e.g., the two, three, four, or five fibers) are together present in an amount of about 0.5% to about 20% w/w of the composition. For example, the two or more fibers are present in an amount of about 0.5% to about 5%, 0.5% to about 10%, 0.5% to about 15%, about 15% to about 20%, about 10% to about 20%, or about 5% to about 20% w/w of the composition. In some embodiments, the two or more fibers are present in an amount of about 0.5% to about 3%, 1% to about 4%, about 2% to about 5%, about 2% to about 6%, about 3% to about 6%, about 3% to about 7%, about 4% to about 7%, about 5% to about 8%, about 5% to about 10%, about 6% to about 9%, about 7% to about 10%, about 8% to about 12%, about 10% to about 15%, or about 12% to about 17% w/w of the composition. For example, about 0.5% to about 1%, about 1% to about 1.5%, about 1% to about 2%, about 1.5% to about 2%, about 1.5% to about 2.5%, about 2% to about 2.5%, about 2% to about 3%, about 2.5% to about 3%, about 2.5% to about 3.5%, about 3% to about 3.5%, about 3% to about 4%, about 3.5% to about 4%, about 3.5% to about 4.5%, about 4% to about 4.5%, about 4% to about 5%, about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, about 14% to about 15%, about 15% to about 16%, about 16% to about 17%, about 17% to about 18%, about 18% to about 19%, or about 19% to about 20% w/w of the composition. In some embodiments, the two or more fibers are present in an amount of about 0.5%, about 0.75%, about 1%, about 1.25%, about 1.5%, about 1.75%, about 2%, about 2.25%, about 2.5%, about 2.75%, about 3%, about 3.25%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20% w/w of the composition.

In some embodiments, each of the first fiber, the second fiber, the third fiber, the fourth fiber, or the fifth fiber is independently present in an amount of about 4% to about 10% w/w of the composition. For example, each of the first fiber, the second fiber, the third fiber, the fourth fiber, or the fifth fiber is independently present in an amount of about 4% to about 5%, about 4% to about 6%, about 4% to about 7%, about 4% to about 8%, about 4% to about 9%, about 9% to about 10%, about 8% to about 10%, about 7% to about 10%, about 6% to about 10%, or about 5% to about 10% w/w of the composition. In some embodiments, each of the first fiber, the second fiber, the third fiber, the fourth fiber, or the fifth fiber is independently present in an amount of about 5% to about 9%, about 5% to about 8%, about 5% to about 7%, about 6% to about 9%, about 6% to about 8%, or about 6% to about 7% w/w of the composition. In some embodiments, each of the first fiber, the second fiber, the third fiber, the fourth fiber, or the fifth fiber is independently present in an amount of about 5%, about 5.5%, about 6%, about 6.5%, about 7%, about 7.5%, about 8%, about 8.5%, about 9%, about 9.5%, or about 10% w/w of the composition.

In some embodiments, each of the first fiber, the second fiber, the third fiber, the fourth fiber, or the fifth fiber is independently present in an amount of about 0.5% to about 8% w/w of the composition. For example, each of the first fiber, the second fiber, the third fiber, the fourth fiber, or the fifth fiber is independently present in an amount of about 0.5% to about 5%, about 2% to about 6%, about 3% to about 7%, or about 5% to about 8% w/w of the composition. In some embodiments, each of the first fiber, the second fiber, the third fiber, the fourth fiber, or the fifth fiber is independently present in an amount of about 0.5% to about 3%, 1% to about 4%, about 2% to about 5%, about 3% to about 6%, about 4% to about 7%, or about 5% to about 8% w/w of the composition. For example, about 0.5% to about 1%, about 0.75% to about 1.25%, about 1% to about 1.5%, about 1% to about 2%, about 1.25% to about 1.75%, about 1.5% to about 2%, about 1.5% to about 2.5%, about 1.75% to about 2.25%, about 2% to about 2.5%, about 2% to about 3%, about 2.25% to about 2.75%, about 2.5% to about 3%, about 2.5% to about 3.5%, about 3% to about 3.5%, about 3% to about 4%, about 3.25% to about 3.75%, about 3.5% to about 4%, about 3.5% to about 4.5%, about 4% to about 4.5%, about 4% to about 5%, about 5% to about 6%, about 6% to about 7%, about 7% to about 8%, about 8% to about 9%, or about 9% to about 10% w/w of the composition. In some embodiments, each of the first fiber, the second fiber, the third fiber, the fourth fiber, or the fifth fiber is independently present in an amount of about 0.5%, about 0.6%, about 0.7%, about 0.75%, about 0.8%, about 0.9%, about 1%, about 1.1%, about 1.2%, about 1.25%, about 1.3%, about 1.4%, about 1.5%, about 1.6%, about 1.7%, about 1.75%, about 1.8%, about 1.9%, about 2%, about 2.25%, about 2.5%, about 2.75%, about 3%, about 3.25%, about 3.5%, about 4%, about 4.5%, about 5%, about 5.5%, about 6%, about 7%, about 8%, about 9%, or about 10% w/w of the composition.

In some embodiments, the composition comprises about 0.5 grams to about 15 grams of the two or more fibers (e.g., the first fiber, the second fiber, the third fiber, the fourth fiber, or the fifth fiber). For example, about 0.5 grams to about 2 grams, about 0.5 grams to about 4 grams, about 0.5 grams to about 6 grams, about 0.5 grams to about 8 grams, about 0.5 grams to about 10 grams, about 0.5 grams to about 12 grams, about 0.5 grams to about 14 grams, about 13 grams to about 15 grams, about 11 grams to about 15 grams, about 9 grams to about 15 grams, about 7 grams to about 15 grams, about 5 grams to about 15 grams, about 3 grams to about 15 grams, or about 1 gram to about 15 grams of the two or more fibers. In some embodiments, the composition comprises about 1 gram to about 3 grams, about 1 gram to about 5 grams, about 1 gram to about 10 grams, about 2 grams to about 4 grams, about 2 grams to about 6 grams, about 3 grams to about 5 grams, about 3 grams to about 6 grams, about 4 grams to about 8 grams, about 4 grams to about 10 grams, about 6 grams to about 10 grams, about 6 grams to about 12 grams, about 8 grams to about 12 grams, or about 8 grams to about 14 grams of the two or more fibers. For example, about 1 gram, 1.5 grams, about 1.8 grams, about 2 grams, about 2.4 grams, about 2.5 grams, about 3 grams, about 3.5 grams, about 3.6 grams, about 4 grams, about 4.2 grams, about 4.5 grams, about 4.8 grams, about 5 grams, about 5.5 grams, about 6 grams, about 6.5 grams, about 7 grams, about 7.5 grams, about 8 grams, about 8.5 grams, about 9 grams, about 9.5 grams, about 10 grams, about 10.5 grams, about 11 grams, about 11.5 grams, about 12 grams, about 12.5 grams, about 13 grams, about 13.5 grams, about 14 grams, about 14.5 grams, or about 15 grams of the two or more fibers.

In some embodiments, the composition comprises about 0.5 grams to about 10 grams of each of the two or more fibers. For example, about 0.5 grams to about 1 gram, about 0.5 grams to about 2 grams, about 0.5 grams to about 3 grams, about 0.5 grams to about 4 grams, about 0.5 grams to about 5 grams, about 0.5 grams to about 6 grams, about 0.5 grams to about 7 grams, about 0.5 grams to about 8 grams, about 0.5 grams to about 9 grams, about 9 grams to about 10 grams, about 8 grams to about 10 grams, about 7 grams to about 10 grams, about 6 grams to about 10 grams, about 5 grams to about 10 grams, about 4 grams to about 10 grams, about 3 grams to about 10 grams, about 2 grams to about 10 grams, or about 1 gram to about 10 grams of each of the two or more fibers. In some embodiments, the composition comprises about 1 gram to about 3 grams, about 1 gram to about 5 grams, about 1 gram to about 6 grams, about 2 grams to about 4 grams, about 2 grams to about 6 grams, about 3 grams to about 5 grams, about 3 grams to about 6 grams, about 4 grams to about 8 grams, or about 4 grams to about 10 grams of each of the two or more fibers. For example, about 1 gram, 1.5 grams, about 1.8 grams, about 2 grams, about 2.4 grams, about 2.5 grams, about 3 grams, about 3.5 grams, about 4 grams, about 4.5 grams, about 5 grams, about 5.5 grams, about 6 grams, about 6.5 grams, about 7 grams, about 7.5 grams, about 8 grams, about 8.5 grams, about 9 grams, about 9.5 grams, or about 10 grams of each of the two or more fibers.

In some embodiments, a fiber is present in the same amount (e.g., % w/w of the composition or grams) as at least one other fiber in the composition. In some embodiments, each fiber is present in the same amount (e.g., % w/w of the composition or grams) as at least one other fiber in the composition. In some embodiments, each fiber is present in the same amount (e.g., % w/w of the composition or grams) as the other fiber(s) in the composition. In some embodiments, each fiber is present in a different amount (e.g., % w/w of the composition or grams) from each other fiber(s) in the composition.

In some embodiments, the compositions as described herein can comprise two fibers present in a ratio of about 1:4 to about 4:1, wherein the ratio is the ratio of the first fiber to the second fiber in the composition. For example, a ratio of about 1:3, about 2:5, about 1:2, about 2:3, about 1:1, about 2:1, about 5:2, about 3:1, or about 3:2 of a first fiber to a second fiber. In some embodiments, the compositions can comprise an additional fiber (e.g., a third fiber, a fourth fiber, etc.) present at a ratio of about 1:4 to about 4:1 relative to another fiber in the composition (e.g., the ratio of a first fiber to a third fiber, a second fiber to a third fiber, a third fiber to a fourth fiber, etc. can be any of the ratios of fibers as described herein). For example, a ratio of about 1:3, about 2:5, about 1:2, about 2:3, about 1:1, about 2:1, about 5:2, about 3:1, or about 3:2 of one fiber to another fiber in the composition.

In some embodiments, the compositions as described herein can comprise a first fiber and a second fiber at a ratio of about 1:4 to about 4:1 first fiber:second fiber. For example, about 1:3, about 2:5, about 1:2, about 2:3, about 1:1, about 2:1, about 5:2, about 3:1, or about 3:2 first fiber:second fiber.

In some embodiments, the first fiber, the second fiber, the third fiber, the fourth fiber, or the fifth fiber is each individually selected from the group consisting of: psyllium, pectin, guar, a lignin, cellulose, hemicellulose, resistant starch, a resistant dextrin, inulin, a chitin, pectin, a beta-glucan, potato starch, and an oligosaccharide. When both a first and a second fiber are present in a composition provided herein, the first and second fibers are different. When a first, a second, and a third fiber are present in a composition provided herein, the first, second, and third fibers are different from each other. When a first, a second, a third, and a fourth fiber are present in a composition provided herein, the first, second, third, and fourth fibers are different from each other. When a first, a second, a third, a fourth, and a fifth fiber are present in a composition provided herein, the first, second, third, fourth, and fifth fibers are different from each other.

In some embodiments, wherein the composition comprises two fibers, the first fiber is an oligosaccharide and the second fiber is selected from the group consisting of: pectin, inulin, psyllium, guar, a lignin, cellulose, hemicellulose, resistant starch, a resistant dextrin, a chitin, a beta-glucan, potato starch, and a combination thereof. In some embodiments, wherein the composition comprises two fibers, the first fiber is an oligosaccharide and the second fiber is pectin. In some embodiments, wherein the composition comprises two fibers, the first fiber is an oligosaccharide and the second fiber is inulin. In some embodiments, wherein the composition comprises two fibers, the first fiber is an oligosaccharide and the second fiber is wheat dextrin. In some embodiments, wherein the composition comprises two fibers, the first fiber is an oligosaccharide and the second fiber is potato starch. In some embodiments, the oligosaccharide is selected from the group consisting of: fructooligosaccharide, maltose, iso-maltose, a maltosyl-iso-malto-oligosaccharide, and a combination thereof. In some embodiments, the oligosaccharide is fructooligosaccharide. In some embodiments, the oligosaccharide is maltose.

In some embodiments, the oligosaccharide is present in an amount of about 5% to about 9% w/w of the composition. In some embodiments, the oligosaccharide is present in an amount of about 6% to about 7% w/w of the composition. In some embodiments, the oligosaccharide is present in an amount of about 6.5% w/w of the composition. In some embodiments, the fiber is present in an amount of about 5% to about 9% w/w of the composition. In some embodiments, the fiber is present in an amount of about 6% to about 7% w/w of the composition. In some embodiments, the fiber is present in an amount of about 6.5% w/w of the composition.

In some embodiments, the composition comprises about 0.5 grams to about 6 grams of an oligosaccharide. In some embodiments, the composition comprises about 0.5 grams to about 6 grams of a fiber is selected from the group consisting of: pectin, inulin, psyllium, guar, a lignin, cellulose, hemicellulose, resistant starch, a resistant dextrin, a chitin, a beta-glucan, potato starch, and a combination thereof. In some embodiments, the composition comprises about 0.5 gramss to about 4 grams of an oligosaccharide. In some embodiments, the composition comprises about 0.5 gramss to about 4 grams of a fiber is selected from the group consisting of: pectin, inulin, psyllium, guar, a lignin, cellulose, hemicellulose, resistant starch, a resistant dextrin, a chitin, a beta-glucan, potato starch, and a combination thereof. In some embodiments, the composition comprises about 0.5 grams to about 2 grams of an oligosaccharide. In some embodiments, the composition comprises about 0.5 grams to about 2 grams of an oligosaccharide. In some embodiments, the composition comprises about 3 grams to about 5 grams of a fiber is selected from the group consisting of: pectin, inulin, psyllium, guar, a lignin, cellulose, hemicellulose, resistant starch, a resistant dextrin, a chitin, a beta-glucan, potato starch, and a combination thereof. In some embodiments, the composition about 0.5 grams, about 1 gram, about 1.5 grams, about 1.8 grams, about 2 grams, about 2.4 grams, about 2.5 grams, about 3 grams, or about 4 grams of an oligosaccharide. In some embodiments, the composition comprises about 0.5 grams, about 1 gram, about 1.5 grams, about 1.8 grams, about 2 grams, about 2.4 grams, about 2.5 grams, about 3 grams, or about 4 grams of a fiber is selected from the group consisting of: pectin, inulin, psyllium, guar, a lignin, cellulose, hemicellulose, resistant starch, a resistant dextrin, a chitin, a beta-glucan, potato starch, and a combination thereof.

In some embodiments, the composition comprises an oligosaccharide and a fiber selected from the group consisting of: pectin, inulin, psyllium, guar, a lignin, cellulose, hemicellulose, resistant starch, a resistant dextrin, a chitin, a beta-glucan, potato starch, and a combination thereof at a ratio of about 1:4 to about 4:1 oligosaccharide:fiber. For example, about 1:3, about 2:5, about 1:2, about 2:3, about 1:1, about 2:1, about 5:2, about 3:1, or about 3:2 an oligosaccharide:fiber. In some embodiments, the oligosaccharide is fructooligosaccharide. In some embodiments, the oligosaccharide is maltose. In some embodiments, the composition comprises a fiber selected from the group consisting of: pectin, inulin, wheat dextrin, potato starch, and a combination thereof.

In some embodiments, wherein the composition comprises two fibers, the first fiber is wheat dextrin and the second fiber is inulin. In some embodiments, wherein the composition comprises two fibers, the first fiber is pectin and the second fiber is inulin. In some embodiments, wherein the composition has two fibers, the first fiber is pectin and the second fiber is potato starch. In some embodiments, wherein the composition has two fibers, the first fiber is potato starch and the second fiber is inulin. In some embodiments, wherein the composition has two fibers, the first fiber is potato starch and the second fiber is wheat dextrin. In some embodiments, wherein the composition has two fibers, the first fiber is pectin and the second fiber is wheat dextrin.

In some embodiments, wherein the composition has four fibers, the first fiber is pectin, the second fiber is guar, the third fiber is inulin, and the fourth fiber is wheat dextrin.

In some embodiments, wherein the composition has four fibers, the first fiber is pectin, the second fiber is guar, the third fiber is potato starch, and the fourth fiber is wheat dextrin.

In some embodiments, a composition as provided herein has two fibers, wherein the two fibers are wheat dextrin and inulin. In some embodiments, the composition comprises wheat dextrin present in an amount of about 5% to about 9% w/w of the composition; and inulin present in an amount of about 5% to about 9% w/w of the composition. In some embodiments, the composition comprises wheat dextrin present in an amount of about 6% to about 7% w/w of the composition; and inulin present in an amount of about 6% to about 7% w/w of the composition. In some embodiments, the composition comprises wheat dextrin present in an amount of about 6.5% w/w of the composition. In some embodiments, the composition comprises inulin present in an amount of about 6.5% w/w of the composition. In some embodiments, the composition comprises wheat dextrin present in an amount of about 2% to about 6% w/w of the composition; and inulin present in an amount of about 2% to about 6% w/w of the composition. In some embodiments, the composition comprises wheat dextrin present in an amount of about 4% w/w of the composition. In some embodiments, the composition comprises inulin present in an amount of about 4% w/w of the composition. In some embodiments, the composition comprises wheat dextrin present in an amount of about 3% w/w of the composition. In some embodiments, the composition comprises inulin present in an amount of about 3% w/w of the composition. In some embodiments, the composition comprises wheat dextrin present in an amount of about 4% w/w of the composition and inulin present in an amount of about 4% w/w of the composition. In some embodiments, the composition comprises wheat dextrin present in an amount of about 4% w/w of the composition and inulin present in an amount of about 3% w/w of the composition. In some embodiments, the composition comprises wheat dextrin present in an amount of about 3% w/w of the composition and inulin present in an amount of about 4% w/w of the composition. In some embodiments, the composition comprises wheat dextrin present in an amount of about 3% w/w of the composition and inulin present in an amount of about 3% w/w of the composition. In some embodiments, the composition further comprises an acid. In some embodiments, the composition further comprises a preservative present in an amount of about 0.01% to about 2% w/w of the composition. In some embodiments, the composition further comprises a sweetener. In some embodiments, the sweetener is maltose.

In some embodiments, the composition comprises about 0.5 grams to about 6 grams of wheat dextrin. In some embodiments, the composition comprises about 0.5 grams to about 6 grams of inulin. In some embodiments, the composition comprises about 1 gram to about 6 grams of wheat dextrin. In some embodiments, the composition comprises about 1 gram to about 6 grams of inulin. In some embodiments, the composition comprises about 1 grams to about 4 grams of wheat dextrin. In some embodiments, the composition comprises about 1 grams to about 4 grams of inulin. In some embodiments, the composition comprises about 0.5 grams to about 2 grams of wheat dextrin. In some embodiments, the composition comprises about 0.5 grams to about 2 grams of inulin. In some embodiments, the composition comprises about 3 grams to about 5 grams of wheat dextrin. In some embodiments, the composition comprises about 3 grams to about 5 grams of inulin. In some embodiments, the composition comprises about 1 gram, about 1.8 grams, about 2 grams, about 2.4 grams, about 2.5 grams, about 3 grams, of about 4 grams of wheat dextrin. In some embodiments, the composition comprises about 0.5 grams, about 1 gram, about 1.5 grams, about 1.8 grams, about 2 grams, about 2.4 grams, about 2.5 grams, about 3 grams, or about 4 grams of inulin.

In some embodiments, the composition comprises wheat dextrin and inulin at a ratio of about 1:4 to about 4:1 wheat dextrin:inulin. For example, about 1:3, about 2:5, about 1:2, about 2:3, about 1:1, about 2:1, about 5:2, about 3:1, or about 3:2 wheat dextrin:inulin.

In some embodiments, a composition as provided herein has two fibers, wherein the two fibers are pectin and inulin. In some embodiments, the composition comprises pectin present in an amount of about 5% to about 9% w/w of the composition; and inulin present in an amount of about 5% to about 9% w/w of the composition. In some embodiments, the composition comprises pectin present in an amount of about 6% to about 7% w/w of the composition; and inulin present in an amount of about 6% to about 7% w/w of the composition. In some embodiments, the composition comprises pectin present in an amount of about 6.5% w/w of the composition. In some embodiments, the composition comprises inulin present in an amount of about 6.5% w/w of the composition. In some embodiments, the composition comprises pectin present in an amount of about 2% to about 6% w/w of the composition; and inulin present in an amount of about 2% to about 6% w/w of the composition. In some embodiments, the composition comprises pectin present in an amount of about 4% w/w of the composition. In some embodiments, the composition comprises inulin present in an amount of about 4% w/w of the composition. In some embodiments, the composition comprises pectin present in an amount of about 3% w/w of the composition. In some embodiments, the composition comprises inulin present in an amount of about 3% w/w of the composition. In some embodiments, the composition comprises pectin present in an amount of about 4% w/w of the composition and inulin present in an amount of about 4% w/w of the composition. In some embodiments, the composition comprises pectin present in an amount of about 4% w/w of the composition and inulin present in an amount of about 3% w/w of the composition. In some embodiments, the composition comprises pectin present in an amount of about 3% w/w of the composition and inulin present in an amount of about 4% w/w of the composition. In some embodiments, the composition comprises pectin present in an amount of about 3% w/w of the composition and inulin present in an amount of about 3% w/w of the composition. In some embodiments, the composition further comprises an acid. In some embodiments, the composition further comprises a preservative present in an amount of about 0.01% to about 2% w/w of the composition. In some embodiments, the composition further comprises a sweetener. In some embodiments, the sweetener is maltose.

In some embodiments, the composition comprises about 0.5 grams to about 6 grams of pectin. In some embodiments, the composition comprises about 0.5 grams to about 6 grams of inulin. In some embodiments, the composition comprises about 1 gram to about 6 grams of pectin. In some embodiments, the composition comprises about 1 gram to about 6 grams of inulin. In some embodiments, the composition comprises about 1 grams to about 4 grams of pectin. In some embodiments, the composition comprises about 1 grams to about 4 grams of inulin. In some embodiments, the composition comprises about 0.5 grams to about 2 grams of pectin. In some embodiments, the composition comprises about 0.5 grams to about 2 grams of inulin. In some embodiments, the composition comprises about 3 grams to about 5 grams of pectin. In some embodiments, the composition comprises about 3 grams to about 5 grams of inulin. In some embodiments, the composition comprises about 0.5 grams, about 1 gram, about 1.5 grams, about 1.8 grams, about 2 grams, about 2.4 grams, about 2.5 grams, about 3 grams, or about 4 grams of pectin. In some embodiments, the composition comprises about 1 gram, about 1.8 grams, about 2 grams, about 2.4 grams, about 2.5 grams, about 3 grams, or about 4 grams of inulin.

In some embodiments, the composition comprises pectin and inulin at a ratio of about 1:4 to about 4:1 pectin:inulin. For example, about 1:3, about 2:5, about 1:2, about 2:3, about 1:1, about 2:1, about 5:2, about 3:1, or about 3:2 pectin:inulin.

In some embodiments, a composition as provided herein has two fibers, wherein the two fibers are pectin and potato starch. In some embodiments, the composition comprises pectin present in an amount of about 5% to about 9% w/w of the composition; and potato starch present in an amount of about 5% to about 9% w/w of the composition. In some embodiments, the composition comprises pectin present in an amount of about 6% to about 7% w/w of the composition; and potato starch present in an amount of about 6% to about 7% w/w of the composition. In some embodiments, the composition comprises pectin present in an amount of about 6.5% w/w of the composition. In some embodiments, the composition comprises potato starch present in an amount of about 6.5% w/w of the composition. In some embodiments, the composition comprises pectin present in an amount of about 2% to about 6% w/w of the composition; and potato starch present in an amount of about 2% to about 6% w/w of the composition. In some embodiments, the composition comprises pectin present in an amount of about 4% w/w of the composition. In some embodiments, the composition comprises potato starch present in an amount of about 4% w/w of the composition. In some embodiments, the composition comprises pectin present in an amount of about 3% w/w of the composition. In some embodiments, the composition comprises potato starch present in an amount of about 3% w/w of the composition. In some embodiments, the composition comprises pectin present in an amount of about 4% w/w of the composition and potato starch present in an amount of about 4% w/w of the composition. In some embodiments, the composition comprises pectin present in an amount of about 4% w/w of the composition and potato starch present in an amount of about 3% w/w of the composition. In some embodiments, the composition comprises pectin present in an amount of about 3% w/w of the composition and potato starch present in an amount of about 4% w/w of the composition. In some embodiments, the composition comprises pectin present in an amount of about 3% w/w of the composition and potato starch present in an amount of about 3% w/w of the composition. In some embodiments, the composition further comprises an acid. In some embodiments, the composition further comprises a preservative present in an amount of about 0.01% to about 2% w/w of the composition. In some embodiments, the composition further comprises a sweetener. In some embodiments, the sweetener is maltose.

In some embodiments, the composition comprises about 0.5 grams to about 6 grams of pectin. In some embodiments, the composition comprises about 0.5 grams to about 6 grams of potato starch. In some embodiments, the composition comprises about 1 gram to about 6 grams of pectin. In some embodiments, the composition comprises about 1 gram to about 6 grams of potato starch. In some embodiments, the composition comprises about 1 grams to about 4 grams of pectin. In some embodiments, the composition comprises about 1 grams to about 4 grams of potato starch. In some embodiments, the composition comprises about 0.5 grams to about 2 grams of pectin. In some embodiments, the composition comprises about 0.5 grams to about 2 grams of potato starch. In some embodiments, the composition comprises about 3 grams to about 5 grams of pectin. In some embodiments, the composition comprises about 3 grams to about 5 grams of potato starch. In some embodiments, the composition comprises about 0.5 grams, about 1 gram, about 1.5 grams, about 1.8 grams, about 2 grams, about 2.4 grams, about 2.5 grams, about 3 grams, or about 4 grams of pectin. In some embodiments, the composition comprises about 1 gram, about 1.8 grams, about 2 grams, about 2.4 grams, about 2.5 grams, about 3 grams, or about 4 grams of potato starch.

In some embodiments, the composition comprises pectin and potato starch at a ratio of about 1:4 to about 4:1 pectin:potato starch. For example, about 1:3, about 2:5, about 1:2, about 2:3, about 1:1, about 2:1, about 5:2, about 3:1, or about 3:2 pectin:potato starch.

In some embodiments, a composition as provided herein has two fibers, wherein the two fibers are potato starch and inulin. In some embodiments, the composition comprises inulin present in an amount of about 5% to about 9% w/w of the composition; and potato starch present in an amount of about 5% to about 9% w/w of the composition. In some embodiments, the composition comprises inulin present in an amount of about 6% to about 7% w/w of the composition; and potato starch present in an amount of about 6% to about 7% w/w of the composition. In some embodiments, the composition comprises inulin present in an amount of about 6.5% w/w of the composition. In some embodiments, the composition comprises potato starch present in an amount of about 6.5% w/w of the composition. In some embodiments, the composition comprises potato starch present in an amount of about 2% to about 6% w/w of the composition; and inulin present in an amount of about 2% to about 6% w/w of the composition. In some embodiments, the composition comprises potato starch present in an amount of about 4% w/w of the composition. In some embodiments, the composition comprises inulin present in an amount of about 4% w/w of the composition. In some embodiments, the composition comprises potato starch present in an amount of about 3% w/w of the composition. In some embodiments, the composition comprises inulin present in an amount of about 3% w/w of the composition. In some embodiments, the composition comprises potato starch present in an amount of about 4% w/w of the composition and inulin present in an amount of about 4% w/w of the composition. In some embodiments, the composition comprises potato starch present in an amount of about 4% w/w of the composition and inulin present in an amount of about 3% w/w of the composition. In some embodiments, the composition comprises potato starch present in an amount of about 3% w/w of the composition and inulin present in an amount of about 4% w/w of the composition. In some embodiments, the composition comprises potato starch present in an amount of about 3% w/w of the composition and inulin present in an amount of about 3% w/w of the composition. In some embodiments, the composition further comprises an acid. In some embodiments, the composition further comprises a preservative present in an amount of about 0.01% to about 2% w/w of the composition. In some embodiments, the composition further comprises a sweetener. In some embodiments, the sweetener is maltose.

In some embodiments, the composition comprises about 0.5 grams to about 6 grams of potato starch. In some embodiments, the composition comprises about 0.5 grams to about 6 grams of inulin. In some embodiments, the composition comprises about 1 gram to about 6 grams of potato starch. In some embodiments, the composition comprises about 1 gram to about 6 grams of inulin. In some embodiments, the composition comprises about 1 grams to about 4 grams of potato starch. In some embodiments, the composition comprises about 1 grams to about 4 grams of inulin. In some embodiments, the composition comprises about 0.5 grams to about 2 grams of potato starch. In some embodiments, the composition comprises about 0.5 grams to about 2 grams of inulin. In some embodiments, the composition comprises about 3 grams to about 5 grams of potato starch. In some embodiments, the composition comprises about 3 grams to about 5 grams of inulin. In some embodiments, the composition comprises about 0.5 grams, about 1 gram, about 1.5 grams, about 1.8 grams, about 2 grams, about 2.4 grams, about 2.5 grams, about 3 grams, or about 4 grams of potato starch. In some embodiments, the composition comprises about 1 gram, about 1.8 grams, about 2 grams, about 2.4 grams, about 2.5 grams, about 3 grams, or about 4 grams of inulin. In some embodiments, the composition comprises potato starch and inulin at a ratio of about 1:4 to about 4:1 potato starch:inulin. For example, about 1:3, about 2:5, about 1:2, about 2:3, about 1:1, about 2:1, about 5:2, about 3:1, or about 3:2 potato starch:inulin.

In some embodiments, a composition as provided herein has two fibers, wherein the two fibers are wheat dextrin and potato starch. In some embodiments, the composition comprises wheat dextrin present in an amount of about 5% to about 9% w/w of the composition; and potato starch present in an amount of about 5% to about 9% w/w of the composition. In some embodiments, the composition comprises wheat dextrin present in an amount of about 6% to about 7% w/w of the composition; and potato starch present in an amount of about 6% to about 7% w/w of the composition. In some embodiments, the composition comprises wheat dextrin present in an amount of about 6.5% w/w of the composition. In some embodiments, the composition comprises potato starch present in an amount of about 6.5% w/w of the composition. In some embodiments, the composition comprises wheat dextrin present in an amount of about 2% to about 6% w/w of the composition; and potato starch present in an amount of about 2% to about 6% w/w of the composition. In some embodiments, the composition comprises wheat dextrin present in an amount of about 4% w/w of the composition. In some embodiments, the composition comprises potato starch present in an amount of about 4% w/w of the composition. In some embodiments, the composition comprises wheat dextrin present in an amount of about 3% w/w of the composition. In some embodiments, the composition comprises potato starch present in an amount of about 3% w/w of the composition. In some embodiments, the composition comprises wheat dextrin present in an amount of about 4% w/w of the composition and potato starch present in an amount of about 4% w/w of the composition. In some embodiments, the composition comprises wheat dextrin present in an amount of about 4% w/w of the composition and potato starch present in an amount of about 3% w/w of the composition. In some embodiments, the composition comprises wheat dextrin present in an amount of about 3% w/w of the composition and potato starch present in an amount of about 4% w/w of the composition. In some embodiments, the composition comprises wheat dextrin present in an amount of about 3% w/w of the composition and potato starch present in an amount of about 3% w/w of the composition. In some embodiments, the composition further comprises an acid. In some embodiments, the composition further comprises a preservative present in an amount of about 0.01% to about 2% w/w of the composition. In some embodiments, the composition further comprises a sweetener. In some embodiments, the sweetener is maltose.

In some embodiments, the composition comprises about 0.5 grams to about 6 grams of wheat dextrin. In some embodiments, the composition comprises about 0.5 grams to about 6 grams of potato starch. In some embodiments, the composition comprises about 1 gram to about 6 grams of wheat dextrin. In some embodiments, the composition comprises about 1 gram to about 6 grams of potato starch. In some embodiments, the composition comprises about 1 grams to about 4 grams of wheat dextrin. In some embodiments, the composition comprises about 1 grams to about 4 grams of potato starch. In some embodiments, the composition comprises about 0.5 grams to about 2 grams of wheat dextrin. In some embodiments, the composition comprises about 0.5 grams to about 2 grams of potato starch. In some embodiments, the composition comprises about 3 grams to about 5 grams of wheat dextrin. In some embodiments, the composition comprises about 3 grams to about 5 grams of potato starch. In some embodiments, the composition comprises about 0.5 grams, about 1 gram, about 1.5 grams, about 1.8 grams, about 2 grams, about 2.4 grams, about 2.5 grams, about 3 grams, or about 4 grams of wheat dextrin. In some embodiments, the composition about 1 gram, about 1.8 grams, about 2 grams, about 2.4 grams, about 2.5 grams, about 3 grams, or about 4 grams of potato starch.

In some embodiments, the composition comprises wheat dextrin and potato starch at a ratio of about 1:4 to about 4:1 wheat dextrin:potato starch. For example, about 1:3, about 2:5, about 1:2, about 2:3, about 1:1, about 2:1, about 5:2, about 3:1, or about 3:2 wheat dextrin:potato starch.

In some embodiments, a composition as provided herein has two fibers, wherein the two fibers are wheat dextrin and pectin. In some embodiments, the composition comprises wheat dextrin present in an amount of about 5% to about 9% w/w of the composition; and pectin present in an amount of about 5% to about 9% w/w of the composition. In some embodiments, the composition comprises wheat dextrin present in an amount of about 6% to about 7% w/w of the composition; and pectin present in an amount of about 6% to about 7% w/w of the composition. In some embodiments, the composition comprises wheat dextrin present in an amount of about 6.5% w/w of the composition. In some embodiments, the composition comprises pectin present in an amount of about 6.5% w/w of the composition. In some embodiments, the composition comprises wheat dextrin present in an amount of about 2% to about 6% w/w of the composition; and pectin present in an amount of about 2% to about 6% w/w of the composition. In some embodiments, the composition comprises wheat dextrin present in an amount of about 4% w/w of the composition. In some embodiments, the composition comprises pectin present in an amount of about 4% w/w of the composition. In some embodiments, the composition comprises wheat dextrin present in an amount of about 3% w/w of the composition. In some embodiments, the composition comprises pectin present in an amount of about 3% w/w of the composition. In some embodiments, the composition comprises wheat dextrin present in an amount of about 4% w/w of the composition and pectin present in an amount of about 4% w/w of the composition. In some embodiments, the composition comprises wheat dextrin present in an amount of about 4% w/w of the composition and pectin present in an amount of about 3% w/w of the composition. In some embodiments, the composition comprises wheat dextrin present in an amount of about 3% w/w of the composition and pectin present in an amount of about 4% w/w of the composition. In some embodiments, the composition comprises wheat dextrin present in an amount of about 3% w/w of the composition and pectin present in an amount of about 3% w/w of the composition. In some embodiments, the composition further comprises an acid. In some embodiments, the composition further comprises a preservative present in an amount of about 0.01% to about 2% w/w of the composition. In some embodiments, the composition further comprises a sweetener. In some embodiments, the sweetener is maltose.

In some embodiments, the composition comprises about 0.5 grams to about 6 grams of wheat dextrin. In some embodiments, the composition comprises about 0.5 grams to about 6 grams of pectin. In some embodiments, the composition comprises about 1 gram to about 6 grams of wheat dextrin. In some embodiments, the composition comprises about 1 gram to about 6 grams of pectin. In some embodiments, the composition comprises about 1 grams to about 4 grams of wheat dextrin. In some embodiments, the composition comprises about 1 grams to about 4 grams of pectin. In some embodiments, the composition comprises about 0.5 grams to about 2 grams of wheat dextrin. In some embodiments, the composition comprises about 0.5 grams to about 2 grams of pectin. In some embodiments, the composition comprises about 3 grams to about 5 grams of wheat dextrin. In some embodiments, the composition comprises about 3 grams to about 5 grams of pectin. In some embodiments, the composition about 0.5 grams, about 1 gram, about 1.5 grams, about 1.8 grams, about 2 grams, about 2.4 grams, about 2.5 grams, about 3 grams, or about 4 grams of wheat dextrin. In some embodiments, the composition comprises about 1 gram, about 1.8 grams, about 2 grams, about 2.4 grams, about 2.5 grams, about 3 grams, or about 4 grams of pectin.

In some embodiments, the composition comprises wheat dextrin and pectin at a ratio of about 1:4 to about 4:1 wheat dextrin:pectin. For example, about 1:3, about 2:5, about 1:2, about 2:3, about 1:1, about 2:1, about 5:2, about 3:1, or about 3:2 wheat dextrin:pectin.

In some embodiments, a composition as provided herein has four fibers, wherein the four fibers are pectin; guar, inulin; and wheat dextrin. In some embodiments, the composition comprises pectin present in an amount of about 0.1% to about 2% w/w of the composition; guar present in an amount of about 0.1% to about 2% w/w of the composition; inulin present in an amount of about 0.1% to about 2% w/w of the composition; and wheat dextrin present in an amount of about 0.1% to about 2% w/w of the composition. In some embodiments, the composition further comprises an acid. In some embodiments, the composition further comprises a preservative present in an amount of about 0.01% to about 2% w/w of the composition. In some embodiments, the composition comprises pectin present in an amount of about 1% w/w of the composition; guar present in an amount of about 1% w/w of the composition; inulin present in an amount of about 1% w/w of the composition; and wheat dextrin present in an amount of about 1% w/w of the composition. In some embodiments, the composition further comprises an acid. In some embodiments, the composition comprises one or more preservatives present in an amount of about 0.01% to about 2% w/w of the composition.

In some embodiments, a composition as provided herein has four fibers, wherein the four fibers are pectin; guar, potato starch; and wheat dextrin. In some embodiments, the composition comprises pectin present in an amount of about 0.1% to about 2% w/w of the composition; guar present in an amount of about 0.1% to about 2% w/w of the composition; potato starch present in an amount of about 0.1% to about 2% w/w of the composition; and wheat dextrin present in an amount of about 0.1% to about 2% w/w of the composition. In some embodiments, the composition further comprises an acid. In some embodiments, the composition comprises one or more preservatives present in an amount of about 0.01% to about 2% w/w of the composition. In some embodiments, the composition comprises pectin present in an amount of about 1% w/w of the composition; guar present in an amount of about 1% w/w of the composition; potato starch present in an amount of about 1% w/w of the composition; and wheat dextrin present in an amount of about 1% w/w of the composition. In some embodiments, the composition further comprises an acid. In some embodiments, the composition comprises one or more preservatives present in an amount of about 0.01% to about 2% w/w of the composition.

Also provided herein is a partially hydrolyzed fiber. In some embodiments, the partially hydrolyzed fiber is a partially hydrolyzed soluble fiber. In some embodiments, the partially hydrolyzed soluble fiber is selected from the group consisting of: partially hydrolyzed psyllium, partially hydrolyzed pectin, partially hydrolyzed wheat dextrin, a partially hydrolyzed beta-glucan, and a combination thereof. In some embodiments, the partially hydrolyzed fiber is selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, a partially hydrolyzed oligosaccharide, partially hydrolyzed potato starch, and a combination thereof. In some embodiments, the partially hydrolyzed fiber is partially hydrolyzed psyllium. In some embodiments, the partially hydrolyzed fiber is partially hydrolyzed pectin.

In some embodiments, the extent of hydrolysis of the two or more fibers can vary. For example, about 1% to about 99% of the hydrolysable chemical bonds may be hydrolyzed. As another example, a fiber can be broken down to different polymer lengths or monosaccharides. In some embodiments, a fiber can be broken down to monosaccharides, disaccharides, or a combination thereof. In some embodiments, a fiber can be broken down to polymer lengths ranging from a monosaccharide to a polymer that is about 99% of the length of the unhydrolyzed polymer. For example, a fiber can be broken down to polymer lengths that are about 2% to about 99%, about 2% to about 50%, about 50% to about 99%, about 10% to about 90%, about 20% to about 80%, or about 25% to about 75% the length of the unhydrolyzed polymer. In some embodiments, a fiber can be broken down to polymer lengths that are about 1% to about 20%, about 10% to about 30%, about 20% to about 40%, about 30 to about 50%, about 40% to about 60%, about 50% to about 70%, about 60% to about 80%, about 70% to about 90%, or about 80% to about 100% the length of the unhydrolyzed polymer. In some embodiments, a fiber can be broken down to polymer lengths that are about 90% to about 99% the length of the unhydrolyzed polymer. For example, about 90% to about 95%, about 95% to about 99%, or about 97% to about 99% the length of the unhydrolyzed polymer. In some embodiments, a fiber can be broken down to polymer lengths that are less than about 90% of the unhydrolyzed polymer length. For example, less than about 80%, about 70%, about 60%, about 50%, about 40%, about 30%, about 20%, or about 10% the length of the unhydrolyzed polymer. The viscosity of a fiber composition may also vary depending on the extent of hydrolysis.

In some embodiments, a composition as provided herein can further comprise one or more of an acid, a preservatives, a sweetener, and a flavoring agent.

Also provided herein are compositions comprising one or more partially hydrolyzed fibers. In some embodiments, the one or more partially hydrolyzed fibers are together present in an amount of about 0.5% to about 20% w/w of the composition. For example, the one or more partially hydrolyzed fibers are present in an amount of about 0.5% to about 5%, 0.5% to about 10%, 0.5% to about 15%, about 15% to about 20%, about 10% to about 20%, or about 5% to about 20% w/w of the composition. In some embodiments, the one or more partially hydrolyzed fibers are present in an amount of about 0.5% to about 3%, 1% to about 4%, about 2% to about 5%, about 2% to about 6%, about 3% to about 6%, about 3% to about 7%, about 4% to about 7%, about 5% to about 8%, about 5% to about 10%, about 6% to about 9%, about 7% to about 10%, about 8% to about 12%, about 10% to about 15%, or about 12% to about 17% w/w of the composition. For example, about 0.5% to about 1%, about 1% to about 1.5%, about 1% to about 2%, about 1.5% to about 2%, about 1.5% to about 2.5%, about 2% to about 2.5%, about 2% to about 3%, about 2.5% to about 3%, about 2.5% to about 3.5%, about 3% to about 3.5%, about 3% to about 4%, about 3.5% to about 4%, about 3.5% to about 4.5%, about 4% to about 4.5%, about 4% to about 5%, about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, about 14% to about 15%, about 15% to about 16%, about 16% to about 17%, about 17% to about 18%, about 18% to about 19%, or about 19% to about 20% w/w of the composition. In some embodiments, the one or more partially hydrolyzed fibers are present in an amount of about 0.5%, about 0.75%, about 1%, about 1.25%, about 1.5%, about 1.75%, about 2%, about 2.25%, about 2.5%, about 2.75%, about 3%, about 3.25%, about 3.5%, about 4%, about 4.5%, about 5%, about 5.5%, about 6%, about 7%, about 8%, about 9%, about 10%, about 11%, about 12%, about 13%, about 14%, about 15%, about 16%, about 17%, about 18%, about 19%, or about 20% w/w of the composition.

In some embodiments, the one or more partially hydrolyzed fibers are selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, partially hydrolyzed potato starch, and a partially hydrolyzed oligosaccharide.

In some embodiments, the one or more partially hydrolyzed fibers is partially hydrolyzed psyllium. In some embodiments, the partially hydrolyzed psyllium is present in an amount of about 0.5% to about 10% w/w of the composition. For example, the partially hydrolyzed psyllium is present in an amount of about 0.5% to about 5%, about 3% to about 7%, or about 5% to about 10% w/w of the composition. In some embodiments, the partially hydrolyzed psyllium is present in an amount of about 0.5% to about 3%, 1% to about 4%, about 2% to about 5%, about 3% to about 6%, about 4% to about 7%, about 5% to about 8%, about 6% to about 9%, or about 7% to about 10% w/w of the composition. For example, about 0.5% to about 1%, about 1% to about 1.5%, about 1% to about 2%, about 1.5% to about 2%, about 1.5% to about 2.5%, about 2% to about 2.5%, about 2% to about 3%, about 2.5% to about 3%, about 2.5% to about 3.5%, about 3% to about 3.5%, about 3% to about 4%, about 3.5% to about 4%, about 3.5% to about 4.5%, about 4% to about 4.5%, about 4% to about 5%, about 5% to about 6%, about 6% to about 7%, about 7% to about 8%, about 8% to about 9%, or about 9% to about 10% w/w of the composition. In some embodiments, the partially hydrolyzed psyllium is present in an amount of about 0.5%, about 0.75%, about 1%, about 1.25%, about 1.5%, about 1.75%, about 2%, about 2.25%, about 2.5%, about 2.75%, about 3%, about 3.25%, about 3.5%, about 4%, about 4.5%, about 5%, about 5.5%, about 6%, about 7%, about 8%, about 9%, or about 10% w/w of the composition.

In some embodiments, the one or more partially hydrolyzed fibers is partially hydrolyzed pectin. In some embodiments, the partially hydrolyzed pectin is present in an amount of about 0.5% to about 10% w/w of the composition. For example, the partially hydrolyzed pectin is present in an amount of about 0.5% to about 5%, about 3% to about 7%, or about 5% to about 10% w/w of the composition. In some embodiments, the partially hydrolyzed pectin is present in an amount of about 0.5% to about 3%, 1% to about 4%, about 2% to about 5%, about 3% to about 6%, about 4% to about 7%, about 5% to about 8%, about 6% to about 9%, or about 7% to about 10% w/w of the composition. For example, about 0.5% to about 1%, about 1% to about 1.5%, about 1% to about 2%, about 1.5% to about 2%, about 1.5% to about 2.5%, about 2% to about 2.5%, about 2% to about 3%, about 2.5% to about 3%, about 2.5% to about 3.5%, about 3% to about 3.5%, about 3% to about 4%, about 3.5% to about 4%, about 3.5% to about 4.5%, about 4% to about 4.5%, about 4% to about 5%, about 5% to about 6%, about 6% to about 7%, about 7% to about 8%, about 8% to about 9%, or about 9% to about 10% w/w of the composition. In some embodiments, the partially hydrolyzed pectin is present in an amount of about 0.5%, about 0.75%, about 1%, about 1.25%, about 1.5%, about 1.75%, about 2%, about 2.25%, about 2.5%, about 2.75%, about 3%, about 3.25%, about 3.5%, about 4%, about 4.5%, about 5%, about 5.5%, about 6%, about 7%, about 8%, about 9%, or about 10% w/w of the composition.

In some embodiments, the composition comprises at least two partially hydrolyzed fibers (e.g., at least a first partially hydrolyzed fiber and a second partially hydrolyzed fiber). In some embodiments, the composition comprises two partially hydrolyzed fibers (e.g., a first partially hydrolyzed fiber and a second partially hydrolyzed fiber). In some embodiments, the composition comprises at least three partially hydrolyzed fibers (e.g., at least a first partially hydrolyzed fiber, a second partially hydrolyzed fiber, and a third partially hydrolyzed fiber). In some embodiments, the composition comprises three partially hydrolyzed fibers (e.g., a first partially hydrolyzed fiber, a second partially hydrolyzed fiber, and a third partially hydrolyzed fiber). In some embodiments, the composition comprises at least four partially hydrolyzed fibers (e.g., at least a first partially hydrolyzed fiber, a second partially hydrolyzed fiber, a third partially hydrolyzed fiber, and a fourth partially hydrolyzed fiber). In some embodiments, the composition comprises four partially hydrolyzed fibers (e.g., a first partially hydrolyzed fiber, a second partially hydrolyzed fiber, a third partially hydrolyzed fiber, and a fourth partially hydrolyzed fiber). In some embodiments, the composition comprises at least five partially hydrolyzed fibers (e.g., at least a first partially hydrolyzed fiber, a second partially hydrolyzed fiber, a third partially hydrolyzed fiber, a fourth partially hydrolyzed fiber, and a fifth partially hydrolyzed fiber). In some embodiments, the composition comprises five partially hydrolyzed fibers (e.g., a first partially hydrolyzed fiber, a second partially hydrolyzed fiber, a third partially hydrolyzed fiber, a fourth partially hydrolyzed fiber, and a fifth partially hydrolyzed fiber).

In some embodiments, each of the first partially hydrolyzed fiber, the second partially hydrolyzed fiber, the third partially hydrolyzed fiber, the fourth partially hydrolyzed fiber, or the fifth partially hydrolyzed fiber is independently present in an amount of about 0.5% to about 8% w/w of the composition. For example, each of the first partially hydrolyzed fiber, the second partially hydrolyzed fiber, the third partially hydrolyzed fiber, the fourth partially hydrolyzed fiber, or the fifth partially hydrolyzed fiber is independently present in an amount of about 0.5% to about 5%, about 2% to about 6%, about 3% to about 7%, or about 5% to about 8% w/w of the composition. In some embodiments, each of the first partially hydrolyzed fiber, the second partially hydrolyzed fiber, the third partially hydrolyzed fiber, the fourth partially hydrolyzed fiber, or the fifth partially hydrolyzed fiber is independently present in an amount of about 0.5% to about 3%, 1% to about 4%, about 2% to about 5%, about 3% to about 6%, about 4% to about 7%, or about 5% to about 8% w/w of the composition. For example, about 0.5% to about 1%, about 0.75% to about 1.25%, about 1% to about 1.5%, about 1% to about 2%, about 1.25% to about 1.75%, about 1.5% to about 2%, about 1.5% to about 2.5%, about 1.75% to about 2.25%, about 2% to about 2.5%, about 2% to about 3%, about 2.25% to about 2.75%, about 2.5% to about 3%, about 2.5% to about 3.5%, about 3% to about 3.5%, about 3% to about 4%, about 3.25% to about 3.75%, about 3.5% to about 4%, about 3.5% to about 4.5%, about 4% to about 4.5%, about 4% to about 5%, about 5% to about 6%, about 6% to about 7%, about 7% to about 8%, about 8% to about 9%, or about 9% to about 10% w/w of the composition. In some embodiments, each of the first partially hydrolyzed fiber, the second partially hydrolyzed fiber, the third partially hydrolyzed fiber, the fourth partially hydrolyzed fiber, or the fifth partially hydrolyzed fiber is independently present in an amount of about 0.5%, about 0.6%, about 0.7%, about 0.75%, about 0.8%, about 0.9%, about 1%, about 1.1%, about 1.2%, about 1.25%, about 1.3%, about 1.4%, about 1.5%, about 1.6%, about 1.7%, about 1.75%, about 1.8%, about 1.9%, about 2%, about 2.25%, about 2.5%, about 2.75%, about 3%, about 3.25%, about 3.5%, about 4%, about 4.5%, about 5%, about 5.5%, about 6%, about 7%, about 8%, about 9%, or about 10% w/w of the composition.

In some embodiments, each partially hydrolyzed fiber is present in the same amount (w/w of the composition) as at least one other partially hydrolyzed fiber in the composition. In some embodiments, each partially hydrolyzed fiber is present in the same amount (w/w of the composition) as the other partially hydrolyzed fiber(s) in the composition. In some embodiments, each partially hydrolyzed fiber is present in a different amount (w/w of the composition) from each other partially hydrolyzed fiber(s) in the composition.

In some embodiments, the first partially hydrolyzed fiber, the second partially hydrolyzed fiber, the third partially hydrolyzed fiber, the fourth partially hydrolyzed fiber, or the fifth partially hydrolyzed fiber is each individually selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, partially hydrolyzed potato starch, and a partially hydrolyzed oligosaccharide. When both a first and a second partially hydrolyzed fiber are present in a composition provided herein, the first and second partially hydrolyzed fibers are different. When a first, a second, and a third partially hydrolyzed fiber are present in a composition provided herein, the first, second, and third partially hydrolyzed fibers are different from each other. When a first, a second, a third, and a fourth partially hydrolyzed fiber are present in a composition provided herein, the first, second, third, and fourth partially hydrolyzed fibers are different from each other. When a first, a second, a third, a fourth, and a fifth partially hydrolyzed fiber are present in a composition provided herein, the first, second, third, fourth, and fifth partially hydrolyzed fibers are different from each other.

In some embodiments, wherein the composition comprises two partially hydrolyzed fibers, the first partially hydrolyzed fiber is partially hydrolyzed pectin, and the second partially hydrolyzed fiber is partially hydrolyzed inulin.

In some embodiments, wherein the composition comprises four partially hydrolyzed fibers, the first partially hydrolyzed fiber is partially hydrolyzed pectin, the second partially hydrolyzed fiber is partially hydrolyzed guar, the third partially hydrolyzed fiber is partially hydrolyzed inulin, and the fourth partially hydrolyzed fiber is partially hydrolyzed wheat dextrin.

In some embodiments, wherein the composition comprises four partially hydrolyzed fibers, the first partially hydrolyzed fiber is partially hydrolyzed pectin, the second partially hydrolyzed fiber is partially hydrolyzed guar, the third partially hydrolyzed fiber is partially hydrolyzed potato starch, and the fourth partially hydrolyzed fiber is partially hydrolyzed wheat dextrin.

In some embodiments, a composition as described herein further comprises an acid. Non-limiting examples of an acid include: sulfuric acid (H₂SO₄), nitric acid (HNO₃), hydrochloric acid (HCl), hydroiodic acid (HI), hydrobromic acid (HBr), perchloric acid (HClO₄), chloric acid (HClO₃), phosphoric acid (H₃PO₄), phosphorous acid, acetic acid, oxalic acid, ascorbic acid, carbonic acid, sulfurous acid, hyposulfurous acid (H₂SO₂), pyrosulfuric acid, dithionous acid, thiosulfurous acid, peroxydisulfuric acid, chlorous acid, hyponitrous acid, carbonous acid, hypocarbonous acid, hypophosphous acid, hypophosphoric acid, hydrophosphoric acid, bromous acid, hypobromous acid, iodous acid, periodic acid, fluorous acid, hypofluorous acid, hydrofluoric acid, chromous acid, perchromic acid, selenic acid, hydronitric acid, molybdic acid, silicofluoric acid, tellurous acid, xenic acid, formic acid, permanganic acid, antimonic acid, silicic acid, arsenic acid, hydroarsenic acid, tetraboric acid, hypooxalous acid, cyanic acid, hydrocyanic acid, uranic acid, tartaric acid, citric acid, malonic acid, phthalic acid, barbituric acid, cinnamic acid, glutaric acid, hexanoic acid, butyric acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, and gallic acid. An acid can be a strong acid (e.g., an acid with an acid dissociation constant (Kₐ) of greater than 1) or a weak acid (e.g., an acid with a Kₐ less than 1). Non-limiting examples of strong acids include HCl, HI, HBr, HClO₄, HClO₃, HNO₃, and H₂SO₄. Non-limiting examples of a weak acid include: phosphorous acid, acetic acid, oxalic acid, phosphoric acid, ascorbic acid, carbonic acid, citric acid, sulfurous acid, tartaric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, and gallic acid.

In some embodiments, the acid is a food-safe acid. Non-limiting examples of food-safe acids include HCl, H₂SO₄, citric acid, malic acid, tartaric acid, acetic acid, oxalic acid, tannic acid, benzoic acid, butyric acid, lactic acid, and phosphoric acid. In some embodiments, food-safe acid is selected from the group consisting of: HCl, H₂SO₄, citric acid, malic acid, tartaric acid, acetic acid, lactic acid, and phosphoric acid.

In some embodiments, the acid is selected from the group consisting of: HCl, HI, HBr, HClO₄, HClO₃, HNO₃, H₂SO₄, phosphoric acid, phosphorous acid, acetic acid, oxalic acid, ascorbic acid, carbonic acid, sulfurous acid, tartaric acid, citric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof. In some embodiments, the acid is phosphoric acid. In some embodiments, the acid is citric acid.

In some embodiments, the acid is a strong acid. In some embodiments, the strong acid is selected from the group consisting of: HCl, HI, HBr, HClO₄, HClO₃, HNO₃, H₂SO₄, and a combination thereof.

In some embodiments, the acid is a weak acid. In some embodiments, the weak acid is selected from the group consisting of: phosphorous acid, acetic acid, oxalic acid, phosphoric acid, ascorbic acid, carbonic acid, citric acid, sulfurous acid, tartaric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof. In some embodiments, the weak acid is phosphoric acid.

In some embodiments, the molarity of the acid is about 0.005 M to about 0.15 M. For example, about 0.005 M to about 0.14 M, about 0.005 M to about 0.13 M, about 0.005 M to about 0.12 M, about 0.005 M to about 0.11 M, about 0.005 M to about 0.1 M, about 0.005 M to about 0.09 M, about 0.005 M to about 0.08 M, about 0.005 M to about 0.07 M, about 0.005 M to about 0.06 M, about 0.005 M to about 0.05 M, about 0.005 M to about 0.04 M, about 0.005 M to about 0.03 M, about 0.005 M to about 0.02 M, about 0.005 M to about 0.01 M, about 0.01 M to about 0.15 M, about 0.01 M to about 0.09 M, about 0.02 M to about 0.08 M, about 0.03 M to about 0.07 M, about 1.4 M to about 0.15 M, about 1.3 M to about 0.15 M, about 1.2 M to about 0.15 M, about 1.1 M to about 0.15 M, about 0.1 M to about 0.15 M, about 0.09 M to about 0.15 M, about 0.08 M to about 0.15 M, about 0.07 M to about 0.15 M, about 0.06 M to about 0.15 M, about 0.05 M to about 0.15 M, about 0.04 M to about 0.15 M, about 0.03 M to about 0.15 M, or about 0.02 M to about 0.15 M. In some embodiments, the molarity of the acid is about 0.005 M to about 0.015 M, about 0.01 M to about 0.02 M, about 0.015 M to about 0.025 M, about 0.02 M to about 0.03 M, about 0.025 M to about 0.035 M, about 0.03 M to about 0.04 M, about 0.035 M to about 0.045 M, about 0.04 M to about 0.05 M, about 0.045 M to about 0.055 M, about 0.05 M to about 0.06 M, about 0.055 M to about 0.065 M, about 0.06 M to about 0.07 M, about 0.065 M to about 0.075 M, about 0.07 M to about 0.08 M, about 0.075 M to about 0.085 M, about 0.08 M to about 0.09 M, about 0.085 M to about 0.095 M, about 0.09 M to about 0.1 M, about 0.095 M to about 0.105 M, about 0.1 M to about 0.11 M, about 0.105 M to about 0.115 M, about 0.11 M to about 0.12 M, about 0.115 M to about 0.125 M, of about 0.12 M to about 0.13 M. For example, about 0.005 M, about 0.0075 M, about 0.01 M, about 0.011, about 0.12, about 0.0125 M, about 0.013, about 0.014, about 0.015 M, about 0.016, about 0.017, about 0.0175 M, about 0.018, about 0.019, about 0.02 M, about 0.0225 M, about 0.025 M, about 0.0275 M, about 0.03 M, about 0.0325 M, about 0.035 M, about 0.0375 M, about 0.04 M, about 0.0425 M, about 0.045 M, about 0.0475 M, about 0.05 M, about 0.0525 M, about 0.055 M, about 0.0575 M, about 0.6 M, about 0.0625 M, about 0.065 M, about 0.0675 M, about 0.07 M, about 0.0725 M, about 0.075 M, about 0.08 M, about 0.09 M, about 0.1 M, about 0.105 M, about 0.11 M, about 0.115 M, about 0.12 M, about 0.125 M, about 0.13 M, about 0.135 M, about 0.14 M, or about 0.145 M.

In some embodiments, the composition has a pH of about 3 to about 5. For example, the composition has a pH of about 3 to about 3.5, about 3 to about 4, about 3 to about 4.5, about 3.5 to about 4, about 3.5 to about 4.5, about 3.5 to about 5, about 4.5 to about 5, or about 4 to about 5. In some embodiments, the composition has a pH of about 3 to about 3.3, about 3.1 to about 3.4, about 3.2 to about 3.5, about 3.3 to about 3.6, about 3.4 to about 3.7, about 3.5 to about 3.8, about 3.6 to about 3.9, about 3.7 to about 4, about 3.8 to about 4.1, about 3.9 to about 4.2, about 4 to about 4.3, about 4.1 to about 4.4, about 4.2 to about 4.5, about 4.3 to about 4.6, about 4.4 to about 4.7, about 4.5 to about 4.8, about 4.6 to about 4.9, or about 4.7 to about 5.0. In some embodiments, the composition has a pH of 3.2 to about 3.4, about 3.3 to about 3.5, about 3.4 to about 3.6, about 3.5 to about 3.7, about 3.6 to about 3.8, about 3.7 to about 3.9, about 3.8 to about 4, about 3.9 to about 4.1, about 4.0 to about 4.2, about 4.1 to about 4.3, or about 4.2 to about 4.4. For example, the composition has a pH of about 3, about 3.1, about 3.2, about 3.3, about 3.4, about 3.5, about 3.6, about 3.7, about 3.8, about 3.9, about 4, about, 4.1, about 4.2, about 4.3, about 4.4, about 4.5, about 4.6, about 4.7, about 4.8, about 4.9, or about 5.

A “preservative” as used herein refers to an agent that protects against decay, discoloration, and/or spoilage. Non-limiting examples of a preservative include ascorbic acid, an ascorbate, a palmitate, citric acid, a benzoate, benzoic acid, a propionate, propionic acid, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, and a tocopherol.

In some embodiments, a composition as described herein can further include a preservative. In some embodiments, the preservative is present in an amount of about 0.01% to about 2% w/w of the composition. In some embodiments, the preservative is present in an amount of about 0.01% to about 1.5%, about 0.01% to about 1.0%, about 0.01% to about 0.5%, about 0.01% to about 0.1%, about 0.01% to about 0.05%, about 1.5% to about 2%, about 1% to about 2%, about 0.5% to about 2%, about 0.1% to about 2%, about 0.05% to about 2%, about 0.01% to about 0.8%, about 0.8% to about 2%, or about 0.1% to about 0.9% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the preservative is present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, ab out 0.38%, about 0.4%, about 0.45%, about 0.5%, about 0.6%, about 0.65%, about 0.7%, about 0.8%, about 0.9%, about 1%, about 1.5%, or about 2% w/w of the composition.

In some embodiments, the preservative is selected from the group consisting of: ascorbic acid, an ascorbate, a palmitate, citric acid, a benzoate, benzoic acid, a propionate, propionic acid, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, a tocopherol, and a combination thereof.

In some embodiments, the preservative is selected from the group consisting of: a palmitate, a benzoate, benzoic acid, a propionate, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, a tocopherol, and a combination thereof.

In some embodiments, the ascorbate is selected from the group consisting of: calcium ascorbate, sodium ascorbate, or a combination thereof. In some embodiments, the palmitate is ascorbyl palmitate. In some embodiments, the benzoate is sodium benzoate. In some embodiments, the propionate is selected from the group consisting of: calcium propionate, sodium propionate, and a combination thereof. In some embodiments, the sorbate is selected from the group consisting of: calcium sorbate, potassium sorbate, sodium sorbate, and a combination of two or more thereof. In some embodiments, the erythorbate is sodium erythorbate. In some embodiments, the nitrite is sodium nitrite. In some embodiments, the bisulfite, sulfite, or metabisulfite is selected from the group consisting of: potassium bisulfite, sodium bisulfite, sodium sulfite, sodium metabisulfite, potassium metabisulfite, and a combination thereof.

In some embodiments, the preservative is a combination of sodium benzoate and potassium sorbate. In some embodiments, the sodium benzoate and potassium sorbate are present in an amount of about 0.01% to about 2% w/w of the composition. In some embodiments, the sodium benzoate and potassium sorbate are present in an amount of about 0.01% to about 1.5%, about 0.01% to about 1.0%, about 0.01% to about 0.5%, about 0.01% to about 0.1%, about 0.01% to about 0.05%, about 1.5% to about 2%, about 1% to about 2%, about 0.5% to about 2%, about 0.1% to about 2%, about 0.05% to about 2%, about 0.01% to about 0.8%, about 0.8% to about 2%, or about 0.1% to about 0.9% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the sodium benzoate and potassium sorbate are present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.5%, or about 2% w/w of the composition.

In some embodiments, the sodium benzoate is present in an amount of about 0.01% to about 1.5%, about 0.01% to about 1.0%, about 0.01% to about 0.5%, about 0.01% to about 0.1%, about 0.01% to about 0.05%, about 1.5% to about 2%, about 1% to about 2%, about 0.5% to about 2%, about 0.1% to about 2%, about 0.05% to about 2%, about 0.01% to about 0.8%, about 0.8% to about 2%, or about 0.1% to about 0.9% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the sodium benzoate is present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.5%, or about 2% w/w of the composition. In some embodiments, the potassium sorbate is present in an amount of about 0.01% to about 1.5%, about 0.01% to about 1.0%, about 0.01% to about 0.5%, about 0.01% to about 0.1%, about 0.01% to about 0.05%, about 1.5% to about 2%, about 1% to about 2%, about 0.5% to about 2%, about 0.1% to about 2%, about 0.05% to about 2%, about 0.01% to about 0.8%, about 0.8% to about 2%, or about 0.1% to about 0.9% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the potassium sorbate is present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.5%, or about 2% w/w of the composition. In some embodiments, the sodium sorbate is present in an amount of 0.05% to about 0.4%, about 0.05% to about 0.2%, about 0.2% to about 0.5%, about 0.15% to about 0.25%, about 0.1% to about 0.3% w/w, or about 0.05% to about 0.15% w/w of the composition and the sodium benzoate is present in an amount of 0.05% to about 0.4%, about 0.05% to about 0.2%, about 0.2% to about 0.5%, about 0.15% to about 0.25%, about 0.1% to about 0.3%, or about 0.05% to about 0.15% w/w of the composition. In some embodiments, the sodium sorbate is present in an amount of about 0.01%, about 0.05%, about 0.06%, about 0.08%, about 0.1%, about 0.12%, about 0.14%, about 0.15%, about 0.16%, about 0.18%, about 0.2%, about 0.22% about 0.25%, about 0.26%, about 0.28%, or about 0.3% w/w of the composition and the sodium benzoate is present in an amount of about 0.01%, about 0.05%, about 0.06%, about 0.08%, about 0.1%, about 0.12%, about 0.14%, about 0.15%, about 0.16%, about 0.18%, about 0.2%, about 0.22% about 0.25%, about 0.26%, about 0.28%, or about 0.3% w/w of the composition. In some embodiments, the sodium sorbate is present in an amount of about 0.2% w/w of the composition and the sodium benzoate is present in an amount of about 0.2% w/w of the composition.

In some embodiments, a composition as described herein can further include a sweetener. Non-limiting examples of a sweetener include sorbitol, sucrose, a saccharin, a cyclamate, aspartame, sucralose, thaumatin, acesulfam K, xylose, ribose, glucose, mannose, galactose, fructose, dextrose, sucrose, maltose, starch or corn syrup, sorbitol, xylitol, mannitol, a steviol glycoside and glycerin. In some embodiments, a steviol glycoside includes stevioside and rebaudioside (e.g., STEVIA® sweetener). A sweetener can be an intense sweetener or a natural sugar.

In some embodiments, the sweetener is present in an amount of about 0.001% to about 20% w/w of the composition. For example, about 0.001% to about 0.05%, about 0.01% to about 0.5%, about 0.1% to about 1.5%, about 1% to about 5%, about 5% to about 10%, about 10% to about 15%, about 15% to about 20% w/w of the composition.

In some embodiments, a composition as described herein can further include a flavoring agent. For example, the composition can further include a flavor enhancer. Non-limiting examples of flavoring agents include a natural flavoring agent (e.g., a substance obtained from a plant or animal raw material by physical, microbiological, or enzymatic processes), a natural fruit flavoring agent (e.g., a substance obtained from a fruit raw material by physical, microbiological, or enzymatic processes), an artificial flavoring agent, an artificial fruit-flavoring agent, and a flavor enhancer. In some embodiments, the flavoring agent is selected from the group consisting of: almond oil, DL-menthol, ethyl acetate, ethyl vanillin, L-menthol, methyl salicylate, peppermint oil, peppermint spirit, thymol, vanillin, cinnamomum, sodium glutamate, eucalyptus, acacia, anise oil, caraway oil, cardamom, cherry syrup, citric acid syrup, clove oil, cocoa, coriander oil, fennel oil, ginger, glycerin, glycerrhiza, honey, lavender oil, lemon oil, mannitol, nutmeg oil, orange oil, orange flower water, raspberry, rose oil, rosemary oil, sarsaparilla syrup, spearmint oil, thyme oil, tolu balsam syrup, wild cherry syrup, and a combination thereof. In some embodiments, the flavoring agent is selected from the group consisting of: almond oil, ethyl vanillin, L-menthol, methyl salicylate, peppermint oil, vanillin, cinnamon, cherry syrup, cocoa, glycerin, honey, lemon oil, and a combination thereof.

In some embodiments, the flavoring agent is present in an amount of about 0.01% to about 2% w/w of the composition.

In some embodiments, a composition as provided herein comprises a partially hydrolyzed psyllium present in amount of about 0.5% to about 10% w/w of the composition; an acid; and one or more preservatives present in an amount of about 0.01% to about 2% w/w of the composition. In some embodiments, the molarity of the acid is about 0.03 M to about 0.07 M. In some embodiments, the pH of the composition is about 3.7 to about 3.9.

In some embodiments, a composition as provided herein comprises a partially hydrolyzed psyllium present in amount of about 0.5% to about 10% w/w of the composition; phosphoric acid; and one or more preservatives present in an amount of about 0.01% to about 2% w/w of the composition. In some embodiments, the molarity of the phosphoric acid is about 0.03 M to about 0.07 M. In some embodiments, the pH of the composition is about 3.7 to about 3.9. In some embodiments, the partially hydrolyzed psyllium is present in amount of about 2% w/w of the composition; and the sodium sorbate and sodium benzoate together are present in an amount of about 0.2% w/w of the composition. In some embodiments, the molarity of the phosphoric acid is 0.05 M. In some embodiments, the pH of the composition is about 3.8.

In some embodiments, a composition as provided herein comprises a partially hydrolyzed pectin present in amount of about 0.5% to about 10% w/w of the composition; an acid; and one or more preservatives present in an amount of about 0.01% to about 2% w/w of the composition. In some embodiments, the molarity of the acid is about 0.05 M to about 0.15 M. In some embodiments, the pH of the composition is about 3.9 to about 4.2.

In some embodiments, a composition as provided herein comprises a partially hydrolyzed pectin present in amount of about 0.5% to about 10% w/w of the composition; citric acid; and one or more preservatives present in an amount of about 0.01% to about 2% w/w of the composition. In some embodiments, the molarity of the citric acid is about 0.05 M to about 0.15 M. In some embodiments, the pH of the composition is about 3.9 to about 4.2. In some embodiments, the partially hydrolyzed pectin is present in amount of about 4% w/w of the composition; and the sodium sorbate and sodium benzoate together are present in an amount of about 0.2% w/w of the composition. In some embodiments, the partially hydrolyzed pectin is present in amount of about 6% w/w of the composition; and the sodium sorbate and sodium benzoate together are present in an amount of about 0.2% w/w of the composition. In some embodiments, the molarity of the acid is 0.013 M. In some embodiments, the pH of the composition is about 3.9 to about 4.2.

In some embodiments, a composition as provided herein comprises two partially hydrolyzed fibers, wherein the two partially hydrolyzed fibers are partially hydrolyzed pectin; and partially hydrolyzed inulin. In some embodiments, the composition comprises partially hydrolyzed pectin present in an amount of about 2% to about 6% w/w of the composition; and partially hydrolyzed inulin present in an amount of about 2% to about 6% w/w of the composition. In some embodiments, the composition comprises partially hydrolyzed pectin present in an amount of about 4% w/w of the composition; and partially hydrolyzed inulin present in an amount of about 4% w/w of the composition. In some embodiments, the composition further comprises an acid. In some embodiments, the composition comprises one or more preservatives present in an amount of about 0.01% to about 2% w/w of the composition.

In some embodiments, a composition as provided herein comprises four partially hydrolyzed fibers, wherein the four partially hydrolyzed fibers are partially hydrolyzed pectin; partially hydrolyzed guar, partially hydrolyzed inulin; and partially hydrolyzed wheat dextrin. In some embodiments, the composition comprises partially hydrolyzed pectin present in an amount of about 0.1% to about 2% w/w of the composition; partially hydrolyzed guar present in an amount of about 0.1% to about 2% w/w of the composition; partially hydrolyzed inulin present in an amount of about 0.1% to about 2% w/w of the composition; and partially hydrolyzed wheat dextrin present in an amount of about 0.1% to about 2% w/w of the composition. In some embodiments, the composition further comprises an acid. In some embodiments, the composition comprises one or more preservatives present in an amount of about 0.01% to about 2% w/w of the composition.

In some embodiments, a composition as provided herein comprises four partially hydrolyzed fibers, wherein the four partially hydrolyzed fibers are partially hydrolyzed pectin; partially hydrolyzed guar, partially hydrolyzed inulin; and partially hydrolyzed wheat dextrin. In some embodiments, the composition comprises partially hydrolyzed pectin present in an amount of about 1% w/w of the composition; partially hydrolyzed guar present in an amount of about 1% w/w of the composition; partially hydrolyzed inulin present in an amount of about 1% w/w of the composition; and partially hydrolyzed wheat dextrin present in an amount of about 1% w/w of the composition. In some embodiments, the composition further comprises an acid. In some embodiments, the composition comprises one or more preservatives present in an amount of about 0.01% to about 2% w/w of the composition.

In some embodiments, a composition as provided herein comprises four partially hydrolyzed fibers, wherein the four partially hydrolyzed fibers are partially hydrolyzed pectin; partially hydrolyzed guar, partially hydrolyzed potato starch; and partially hydrolyzed wheat dextrin. In some embodiments, the composition comprises partially hydrolyzed pectin present in an amount of about 0.1% to about 2% w/w of the composition; partially hydrolyzed guar present in an amount of about 0.1% to about 2% w/w of the composition; partially hydrolyzed potato starch present in an amount of about 0.1% to about 2% w/w of the composition; and partially hydrolyzed wheat dextrin present in an amount of about 0.1% to about 2% w/w of the composition. In some embodiments, the composition further comprises an acid. In some embodiments, the composition comprises one or more preservatives present in an amount of about 0.01% to about 2% w/w of the composition.

In some embodiments, a composition as provided herein comprises four partially hydrolyzed fibers, wherein the four partially hydrolyzed fibers are partially hydrolyzed pectin; partially hydrolyzed guar, partially hydrolyzed potato starch; and partially hydrolyzed wheat dextrin. In some embodiments, the composition comprises partially hydrolyzed pectin present in an amount of about 1% w/w of the composition; partially hydrolyzed guar present in an amount of about 1% w/w of the composition; partially hydrolyzed potato starch present in an amount of about 1% w/w of the composition; and partially hydrolyzed wheat dextrin present in an amount of about 1% w/w of the composition. In some embodiments, the composition further comprises an acid. In some embodiments, the composition comprises one or more preservatives present in an amount of about 0.01% to about 2% w/w of the composition.

In some embodiments, the one or more fibers can be broken down to monosaccharides, disaccharides, or a combination thereof. In some embodiments, the one or more fibers can be broken down to polymer lengths ranging from a monosaccharide to a polymer that is about 99% of the length of the unhydrolyzed polymer. For example, the one or more fibers can be broken down to polymer lengths that are about 2% to about 99%, about 2% to about 50%, about 50% to about 99%, about 10% to about 90%, about 20% to about 80%, or about 25% to about 75% the length of the unhydrolyzed polymer. In some embodiments, the one or more fibers can be broken down to polymer lengths that are about 10% to about 30%, about 20% to about 40%, about 30 to about 50%, about 40% to about 60%, about 50% to about 70%, about 60% to about 80%, about 70% to about 90%, or about 80% to about 100% the length of the unhydrolyzed polymer. In some embodiments, the one or more fibers can be broken down to polymer lengths that are about 90% to about 99% the length of the unhydrolyzed polymer. For example, about 90% to about 95%, about 95% to about 99%, or about 97% to about 99% the length of the unhydrolyzed polymer. In some embodiments, the one or more fibers can be broken down to polymer lengths that are less than about 90% of the unhydrolyzed polymer length. For example, less than about 80%, about 70%, about 60%, about 50%, about 40%, about 30%, about 20%, or about 10% the length of the unhydrolyzed polymer. The viscosity of the partially hydrolyzed fiber composition may also vary depending on the extent of hydrolysis.

In some embodiments, a composition as provided herein is a liquid. For example, the composition may be an aqueous solution comprising one or more partially hydrolyzed fibers. In some embodiments, the liquid composition is selected from the group consisting of: a solution, a syrup, a suspension, and a concentrate. In some embodiments, a liquid composition as provided herein is carbonated. In some embodiments, the partially hydrolyzed fiber composition is texturized.

In some embodiments, a composition as provided herein is a solid composition (e.g., a powder).

In some embodiments, a composition as provided herein is clear. In some embodiments, a composition as provided herein is provided as a suspension (e.g., a suspension of one or more partially hydrolyzed fibers in an aqueous solution). In some embodiments, a suspension may be cloudy.

In some embodiments, a composition as provided herein is added to a food. For example, the partially hydrolyzed fiber composition may be added to cereal, yogurt, pudding, and gummies. In some embodiments, the partially hydrolyzed fiber composition is added to a beverage. For example, the partially hydrolyzed fiber composition may be added to milk, juice, or lemonade.

### Kits

This disclosure also provides kits for a dietary fiber combination, e.g., a kit, comprising i) a first fiber; and ii) a second fiber. In some embodiments, the kit comprises i) a first fiber; ii) a second fiber; and iii) a third fiber. In some embodiments, the kit comprises i) a first fiber; ii) a second fiber; iii) a third fiber, and iv) a fourth fiber. In some embodiments, the fibers (e.g., a first fiber and a second fiber) are present as a composition. In some embodiments, each fiber is present as an independent composition from each other fiber.

In some embodiments, the kit comprises about 2% to about 6% w/w of the composition of the first fiber. In some embodiments, the kit comprises about 2% to about 6% w/w of the composition of the second fiber. In some embodiments, the kit comprises about 4% w/w of the composition of the first fiber. In some embodiments, the kit comprises about 4% w/w of the composition of the second fiber. In some embodiments, the kit comprises about 3% w/w of the composition of the first fiber. In some embodiments, the kit comprises about 3% w/w of the composition of the second fiber.

In some embodiments, the kit comprises about 1 gram to about 6 grams of the first fiber; and about 1 gram to about 6 grams of the second fiber. In some embodiments, the kit comprises about 2 grams to about 3 grams of the first fiber; and about 2 grams to about 3 grams of the second fiber. In some embodiments, the kit comprises about 2.4 grams of the first fiber; and about 2.4 grams of the second fiber. In some embodiments, the kit comprises about 2.5 grams of the first fiber; and about 2.5 grams of the second fiber. In some embodiments, the kit comprises about 2 grams to about 6 grams of the first fiber. In some embodiments, the kit comprises about 2 grams to about 6 grams of the second fiber. In some embodiments, the kit comprises about 4 grams of the first fiber. In some embodiments, the kit comprises about 4 grams of the second fiber. In some embodiments, the kit comprises about 1 gram, 1.5 grams, about 2 grams, about 2.1 grams, about 2.2 grams, about 2.3 grams, about 2.4 grams, about 2.5 grams, about 2.6 grams, about 2.7 grams, about 2.8 grams, about 2.9 grams, about 3.1 grams, about 3.2 grams, about 3.3 grams, about 3.4 grams, about 3.5 grams, about 3.6 grams, about 3.7 grams, about 3.8 grams, about 3.9 grams, about 4 grams, about 4.1 grams, about 4.2 grams, about 4.3 grams, about 4.4 grams, about 4.5 grams, about 4.6 grams, about 4.7 grams, about 4.8 grams, about 4.9 grams, or about 5 grams, of wheat dextrin. In some embodiments, the composition comprises about 1 gram, 1.5 grams, about 2 grams, about 2.1 grams, about 2.2 grams, about 2.3 grams, about 2.4 grams, about 2.5 grams, about 2.6 grams, about 2.7 grams, about 2.8 grams, about 2.9 grams, about 3.1 grams, about 3.2 grams, about 3.3 grams, about 3.4 grams, about 3.5 grams, about 3.6 grams, about 3.7 grams, about 3.8 grams, about 3.9 grams, about 4 grams, about 4.1 grams, about 4.2 grams, about 4.3 grams, about 4.4 grams, about 4.5 grams, about 4.6 grams, about 4.7 grams, about 4.8 grams, about 4.9 grams, or about 5 grams of the first fiber. In some embodiments, the kit comprises about 1 gram, 1.5 grams, about 2 grams, about 2.1 grams, about 2.2 grams, about 2.3 grams, about 2.4 grams, about 2.5 grams, about 2.6 grams, about 2.7 grams, about 2.8 grams, about 2.9 grams, about 3.1 grams, about 3.2 grams, about 3.3 grams, about 3.4 grams, about 3.5 grams, about 3.6 grams, about 3.7 grams, about 3.8 grams, about 3.9 grams, about 4 grams, about 4.1 grams, about 4.2 grams, about 4.3 grams, about 4.4 grams, about 4.5 grams, about 4.6 grams, about 4.7 grams, about 4.8 grams, about 4.9 grams, or about 5 grams, of wheat dextrin. In some embodiments, the composition comprises about 1 gram, 1.5 grams, about 2 grams, about 2.1 grams, about 2.2 grams, about 2.3 grams, about 2.4 grams, about 2.5 grams, about 2.6 grams, about 2.7 grams, about 2.8 grams, about 2.9 grams, about 3.1 grams, about 3.2 grams, about 3.3 grams, about 3.4 grams, about 3.5 grams, about 3.6 grams, about 3.7 grams, about 3.8 grams, about 3.9 grams, about 4 grams, about 4.1 grams, about 4.2 grams, about 4.3 grams, about 4.4 grams, about 4.5 grams, about 4.6 grams, about 4.7 grams, about 4.8 grams, about 4.9 grams, or about 5 grams of the second fiber.

In some embodiments, the kit comprises a first fiber and a second fiber at a ratio of about 1:4 to about 4:1 first fiber:second fiber. For example, about 1:3, about 2:5, about 1:2, about 2:3, about 1:1, about 2:1, about 5:2, about 3:1, or about 3:2 first fiber:second fiber.

In some embodiments, the first fiber is wheat dextrin and the second fiber is inulin. In some embodiments, the first fiber is pectin and the second fiber is inulin. In some embodiments, the first fiber is pectin and the second fiber is potato starch. In some embodiments, the first fiber is potato starch and the second fiber is inulin. In some embodiments, the first fiber is wheat dextrin and the second fiber is potato starch. In some embodiments, the first fiber is wheat dextrin and the second fiber is pectin.

In some embodiments, the remaining part of a composition is water or buffered water. In some embodiments, the composition is a liquid composition. In some embodiments, the liquid composition is selected from the group consisting of: a solution, a syrup, a suspension, and a concentrate.

The kit can further comprise instructions for the administration of the fibers (e.g., the first fiber and second fiber).

### Methods for Preparing Partially Hydrolyzed Fibers

Also provided herein are methods for preparing a partially hydrolyzed fiber. In some embodiments, the method comprises contacting one or more fibers with an effective amount of a hydrolyzing agent. Non-limiting examples of a hydrolyzing agent include a hydrolytic enzyme and an acid. Non-limiting examples of a hydrolytic enzyme includes a glycosidase such as an amylase. One of skill in the art will recognize that any hydrolytic enzyme that can hydrolyze one or more bonds of a fiber as described herein can be used.

In some embodiments, the method comprises contacting one or more fibers with an effective amount of a hydrolyzing agent to form a mixture. In some embodiments, the method includes incubating the mixture at about room temperature to form an incubated mixture. In some embodiments, the method includes incubating the mixture at a temperature of about 50° C. to about 85° C. to form a heated and incubated mixture. In some embodiments, the method comprises contacting one or more fibers with an effective amount of a hydrolyzing agent to form a mixture and incubating the mixture for a period of time at a temperature of about 60° C. to about 75° C. to form a heated and incubated mixture. In some embodiments, the method comprises contacting one or more fibers with an effective amount of a hydrolyzing agent to form a mixture and incubating the mixture for a period of time at a temperature of about 65° C. to about 70° C. to form a heated and incubated mixture.

In some embodiments, the method comprises contacting one or more fibers with an effective amount of a hydrolyzing agent to form a mixture and incubating the mixture for a period of time to form an incubated mixture. In some embodiments, the period of time is about 0.1 to about 15 hours, about 0.1 to about 10 hours, about 0.1 to about 5 hours, about 1 to about 15 hours, about 1 to about 10 hours, about 1 to about 5 hours, about 5 to about 15 hours, about 0.1 to about 2 hours, about 10 to about 15 hours, or about 5 to about 8 hours. In some embodiments, the period of time is about 6 to about 7 hours. In some embodiments, the period of time is about 0.1 hours, about 0.2 hours, about 0.5 hours, about 1 hour, about 2 hours, about 3 hours, about 4 hours, about 5 hours, about 5.5 hours, about 6 hours, about 6.5 hours, about 7 hours, about 7.5 hours, about 8 hours, or about 8.5 hours. In some embodiments, the method comprises contacting one or more fibers with an effective amount of a hydrolyzing agent to form a mixture and incubating the mixture for a period of time of about 6.5 hours at a temperature of about 65° C. to about 70° C. to form a heated and incubated mixture.

In some embodiments, the pH of the mixture is about 1 to about 4, about 2 to about 4, about 3 to about 4, about 1 to about 4, about 1 to about 2, or about 2 to about 3. In some embodiments, the pH of the mixture is about 1, about 1.2, about 1.3, about 1.4, about 1.5, about 1.6, about 1.7, about 1.8, about 1.9, about 2, about 2.1, about 2.2, about 2.3, about 2.4, about 2.5, about 3, or about 4.

In some embodiments, the hydrolyzing agent is an acid. In some embodiments, the molarity of the acid in the mixture is about 0.005 M to about 0.15 M. For example, about 0.005 M to about 0.14 M, about 0.005 M to about 0.13 M, about 0.005 M to about 0.12 M, about 0.005 M to about 0.11 M, about 0.005 M to about 0.1 M, about 0.005 M to about 0.09 M, about 0.005 M to about 0.08 M, about 0.005 M to about 0.07 M, about 0.005 M to about 0.06 M, about 0.005 t M o about 0.05 M, about 0.005 M to about 0.04 M, about 0.005 M to about 0.03 M, about 0.005 M to about 0.02 M, about 0.005 M to about 0.01 M, about 0.01 M to about 0.15 M, about 0.01 M to about 0.09 M, about 0.02 M to about 0.08 M, about 0.03 M to about 0.07 M, about 1.4 M to about 0.15 M, about 1.3 M to about 0.15 M, about 1.2 M to about 0.15 M, about 1.1 M to about 0.15 M, about 0.1 M to about 0.15 M, about 0.09 M to about 0.15 M, about 0.08 M to about 0.15 M, about 0.07 M to about 0.15 M, about 0.06 M to about 0.15 M, about 0.05 M to about 0.15 M, about 0.04 M to about 0.15 M, about 0.03 M to about 0.15 M, or about 0.02 M to about 0.15 M. In some embodiments, the molarity of the acid is about 0.005 M to about 0.015 M, about 0.01 M to about 0.02 M, about 0.015 M to about 0.025 M, about 0.02 M to about 0.03 M, about 0.025 M to about 0.035 M, about 0.03 M to about 0.04 M, about 0.035 M to about 0.045 M, about 0.04 M to about 0.05 M, about 0.045 M to about 0.055 M, about 0.05 M to about 0.06 M, about 0.055 M to about 0.065 M, about 0.06 M to about 0.07 M, about 0.065 M to about 0.075 M, about 0.07 M to about 0.08 M, about 0.075 M to about 0.085 M, about 0.08 M to about 0.09 M, about 0.085 M to about 0.095 M, or about 0.09 M to about 0.1 M, about 0.095 M to about 0.105 M, about 0.1 M to about 0.11 M, about 0.105 M to about 0.115 M, about 0.11 M to about 0.12 M, about 0.115 M to about 0.125 M, of about 0.12 M to about 0.13 M. For example, about 0.005 M, about 0.0075 M, about 0.01 M, about 0.011, about 0.12, about 0.0125 M, about 0.013, about 0.014, about 0.015 M, about 0.016, about 0.017, about 0.0175 M, about 0.018, about 0.019, about 0.02 M, about 0.0225 M, about 0.025 M, about 0.0275 M, about 0.03 M, about 0.0325 M, about 0.035 M, about 0.0375 M, about 0.04 M, about 0.0425 M, about 0.045 M, about 0.0475 M, about 0.05 M, about 0.0525 M, about 0.055 M, about 0.0575 M, about 0.6 M, about 0.0625 M, about 0.065 M, about 0.0675 M, about 0.07 M, about 0.0725 M, about 0.075 M, about 0.08 M, about 0.09 M, or about 0.1 M, about 0.105 M, about 0.11 M, about 0.115 M, about 0.12 M, about 0.125 M, about 0.13 M, about 0.135 M, about 0.14 M, or about 0.145 M.

In some embodiments, the method further comprises cooling the heated mixture to form a partially hydrolyzed fiber mixture. In some embodiments, the method further comprises cooling the heated mixture rapidly to form a partially hydrolyzed fiber mixture. In some embodiments, the method further comprises cooling the heated mixture with mixing to form a partially hydrolyzed fiber mixture. In some embodiments, the mixture is cooled to about room temperature.

In some embodiments, the method further comprises cooling the heated and incubated mixture to form a partially hydrolyzed fiber mixture. In some embodiments, the method further comprises cooling the heated and incubated mixture rapidly to form a partially hydrolyzed fiber mixture. In some embodiments, the method further comprises cooling the heated and incubated mixture with mixing to form a partially hydrolyzed fiber mixture. In some embodiments, the mixture is cooled to about room temperature.

In some embodiments, one or more preservatives are added to the partially hydrolyzed fiber mixture.

In some embodiments, the method further comprises adjusting the pH of the partially hydrolyzed fiber mixture to a pH of about 3 to about 5. In some embodiments, the method further comprises adjusting the pH of the partially hydrolyzed fiber mixture to a pH of about 3.5 to about 4. In some embodiments, the method further comprises adjusting the pH of the partially hydrolyzed fiber mixture to a pH of about 3.8. In some embodiments, the method further comprises adjusting the pH of the partially hydrolyzed fiber mixture to a pH of about 3 to about 5. In some embodiments, the method further comprises adjusting the pH of the partially hydrolyzed fiber mixture to a pH of about 3.9 to about 4.2.

In some embodiments, the method further comprises adding one or more sweeteners. In some embodiments, the method further comprises adding one or more flavoring agents.

Also provided herein is a method for preparing a partially hydrolyzed fiber comprising:

contacting one or more fibers with an effective amount of a hydrolyzing agent to form a mixture; and

incubating the mixture for a period of time to form a partially hydrolyzed fiber mixture. In some embodiments, the mixture is incubated at about room temperature. In some embodiments, the mixture is stirred during the period of time.

Also provided herein is a method for preparing a partially hydrolyzed fiber comprising:

contacting one or more fibers with an effective amount of a hydrolyzing agent to form a mixture;

heating the mixture to about 50° C. to about 80° C. to form a heated mixture;

incubating the heated mixture at about 50° C. to about 80° C. for a period of time to form a heated and incubated mixture;

and cooling the heated and incubated mixture to form a partially hydrolyzed fiber mixture.

In some embodiments, the pH of the mixture is about 1 to about 4, about 2 to about 4, about 3 to about 4, about 1 to about 4, about 1 to about 2, or about 2 to about 3. In some embodiments, the pH of the mixture is about 1, about 1.2, about 1.3, about 1.4, about 1.5, about 1.6, about 1.7, about 1.8, about 1.9, about 2, about 2.1, about 2.2, about 2.3, about 2.4, about 2.5, about 3, or about 4.

In some embodiments, the hydrolyzing agent is an acid. In some embodiments, the molarity of the acid in the mixture is about 0.005 to about 0.1 M. For example, about 0.005 to about 0.09 M, about 0.005 to about 0.08 M, about 0.005 to about 0.07 M, about 0.005 to about 0.06 M, about 0.005 to about 0.05 M, about 0.005 to about 0.04 M, about 0.005 to about 0.03 M, about 0.005 to about 0.02 M, about 0.005 to about 0.01 M, about 0.01 to about 0.1 M, about 0.01 to about 0.09 M, about 0.02 to about 0.08 M, about 0.03 to about 0.07 M, about 0.09 to about 0.1 M, about 0.08 to about 0.1 M, about 0.07 to about 0.1 M, about 0.06 to about 0.1 M, about 0.05 to about 0.1 M, about 0.04 to about 0.1 M, about 0.03 to about 0.1 M, or about 0.02 to about 0.1 M. In some embodiments, the molarity of the acid is about 0.005 M to about 0.015 M, about 0.01 M to about 0.02 M, about 0.015 M to about 0.025 M, about 0.02 M to about 0.03 M, about 0.025 M to about 0.035 M, about 0.03 M to about 0.04 M, about 0.035 M to about 0.045 M, about 0.04 M to about 0.05 M, about 0.045 M to about 0.055 M, about 0.05 M to about 0.06 M, about 0.055 M to about 0.065 M, about 0.06 M to about 0.07 M, about 0.065 M to about 0.075 M, about 0.07 M to about 0.08 M, about 0.075 M to about 0.085 M, about 0.08 M to about 0.09 M, about 0.085 M to about 0.095 M, or about 0.09M to about 0.1 M. For example, about 0.005 M, about 0.0075 M, about 0.01 M, about 0.0125 M, about 0.015 M, about 0.0175 M, about 0.02 M, about 0.0225 M, about 0.025 M, about 0.0275 M, about 0.03 M, about 0.0325 M, about 0.035 M, about 0.0375 M, about 0.04 M, about 0.0425 M, about 0.045 M, about 0.0475 M, about 0.05 M, about 0.0525 M, about 0.055 M, about 0.0575 M, about 0.6 M, about 0.0625 M, about 0.065 M, about 0.0675 M, about 0.07 M, about 0.0725 M, about 0.075 M, about 0.08 M, about 0.09 M, or about 0.1 M.

In some embodiments, the mixture is heated to about 60° C. to about 75° C. In some embodiments, the mixture is heated to about 65° C. to about 70° C. In some embodiments, the heated mixture is incubated at a temperature of about 60° C. to about 75° C. In some embodiments, the heated mixture is incubated at a temperature of about 65° C. to about 70° C. In some embodiments, the mixture is heated rapidly.

In some embodiments, the method further comprises stirring the mixture during heating. In some embodiments, the method further comprises stirring the heated mixture during the period of time.

In some embodiments, water is added to the heated mixture to return to the original, pre-heated weight. In some embodiments, the heated mixture is cooled rapidly. In some embodiments, the method further comprises stirring the heated mixture during cooling. In some embodiments, the mixture is cooled to about room temperature.

In some embodiments, the method further comprises adding one or more preservatives to the partially hydrolyzed fiber mixture.

In some embodiments, the method further comprises filtering the partially hydrolyzed fiber mixture.

In some embodiments, the method further comprises adjusting the pH of the partially hydrolyzed fiber mixture to a pH of about 3 to about 5. In some embodiments, the method further comprises adjusting the pH of the partially hydrolyzed fiber mixture to a pH of about 3.5 to about 4. In some embodiments, the method further comprises adjusting the pH of the partially hydrolyzed fiber mixture to a pH of about 3.8.

In some embodiments, the method further comprises adding one or more sweeteners to the partially hydrolyzed fiber mixture.

In some embodiments, the method further comprises adding one or more flavoring agents to the partially hydrolyzed fiber mixture.

In some embodiments, the one or more fibers are present in an amount of about 0.5% to about 10% w/w of the mixture. For example, the one or more fibers are present in an amount of about 0.5% to about 5%, about 3% to about 7%, or about 5% to about 10% w/w of the mixture. In some embodiments, the one or more fibers are present in an amount of about 0.5% to about 3%, 1% to about 4%, about 2% to about 5%, about 3% to about 6%, about 4% to about 7%, about 5% to about 8%, about 6% to about 9%, or about 7% to about 10% w/w of the mixture. For example, about 0.5% to about 1%, about 1% to about 1.5%, about 1% to about 2%, about 1.5% to about 2%, about 1.5% to about 2.5%, about 2% to about 2.5%, about 2% to about 3%, about 2.5% to about 3%, about 2.5% to about 3.5%, about 3% to about 3.5%, about 3% to about 4%, about 3.5% to about 4%, about 3.5% to about 4.5%, about 4% to about 4.5%, about 4% to about 5%, about 5% to about 6%, about 6% to about 7%, about 7% to about 8%, about 8% to about 9%, or about 9% to about 10% w/w of the mixture. In some embodiments, the one or more fibers are present in an amount of about 0.5%, about 0.75%, about 1%, about 1.25%, about 1.5%, about 1.75%, about 2%, about 2.25%, about 2.5%, about 2.75%, about 3%, about 3.25%, about 3.5%, about 4%, about 4.5%, about 5%, about 5.5%, about 6%, about 7%, about 8%, about 9%, or about 10% w/w of the mixture.

In some embodiments, the fiber is selected from the group consisting of: psyllium, pectin, guar, a lignin, cellulose, hemicellulose, resistant starch, a resistant dextrin, inulin, a chitin, pectin, a beta-glucan, an oligosaccharide, potato starch, and a combination of two or more thereof.

In some embodiments, the fiber is psyllium. In some embodiments, the psyllium is present in an amount of about 0.5% to about 10% w/w of the mixture. For example, the psyllium is present in an amount of about 0.5% to about 5%, about 3% to about 7%, or about 5% to about 10% w/w of the mixture. In some embodiments, the psyllium is present in an amount of about 0.5% to about 3%, 1% to about 4%, about 2% to about 5%, about 3% to about 6%, about 4% to about 7%, about 5% to about 8%, about 6% to about 9%, or about 7% to about 10% w/w of the mixture. For example, about 0.5% to about 1%, about 1% to about 1.5%, about 1% to about 2%, about 1.5% to about 2%, about 1.5% to about 2.5%, about 2% to about 2.5%, about 2% to about 3%, about 2.5% to about 3%, about 2.5% to about 3.5%, about 3% to about 3.5%, about 3% to about 4%, about 3.5% to about 4%, about 3.5% to about 4.5%, about 4% to about 4.5%, about 4% to about 5%, about 5% to about 6%, about 6% to about 7%, about 7% to about 8%, about 8% to about 9%, or about 9% to about 10% w/w of the mixture. In some embodiments, the psyllium is present in an amount of about 0.5%, about 0.75%, about 1%, about 1.25%, about 1.5%, about 1.75%, about 2%, about 2.25%, about 2.5%, about 2.75%, about 3%, about 3.25%, about 3.5%, about 4%, about 4.5%, about 5%, about 5.5%, about 6%, about 7%, about 8%, about 9%, about 10% w/w of the mixture.

In some embodiments, the fiber is pectin. In some embodiments, the pectin is present in an amount of about 0.5% to about 10% w/w of the mixture. For example, the pectin is present in an amount of about 0.5% to about 5%, about 1% to about 7%, about 3% to about 6%, or about 4% to about 8% w/w of the mixture. In some embodiments, the pectin is present in an amount of about 0.5% to about 3%, 1% to about 4%, about 2% to about 5%, about 3% to about 6%, about 4% to about 7%, about 5% to about 8%, about 6% to about 9%, or about 7% to about 10% w/w of the mixture. For example, about 0.5% to about 1%, about 1% to about 1.5%, about 1% to about 2%, about 1.5% to about 2%, about 1.5% to about 2.5%, about 2% to about 2.5%, about 2% to about 3%, about 2.5% to about 3%, about 2.5% to about 3.5%, about 3% to about 3.5%, about 3% to about 4%, about 3.5% to about 4%, about 3.5% to about 4.5%, about 4% to about 4.5%, about 4% to about 5%, about 5% to about 6%, about 6% to about 7%, about 7% to about 8%, about 8% to about 9%, or about 9% to about 10% w/w of the mixture. In some embodiments, the pectin is present in an amount of about 0.5%, about 0.75%, about 1%, about 1.25%, about 1.5%, about 1.75%, about 2%, about 2.25%, about 2.5%, about 2.75%, about 3%, about 3.25%, about 3.5%, about 4%, about 4.5%, about 5%, about 5.5%, about 6%, about 7%, about 8%, about 9%, about 10% w/w of the mixture.

In some embodiments, the hydrolyzing agent is an acid. In some embodiments, the acid is selected from the group consisting of: HCl, HI, HBr, HClO₄, HClO₃, HNO₃, H₂SO₄, phosphoric acid, phosphorous acid, acetic acid, oxalic acid, ascorbic acid, carbonic acid, sulfurous acid, tartaric acid, citric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof. In some embodiments, the acid is phosphoric acid. In some embodiments, the acid is citric acid.

In some embodiments, the acid is a strong acid. In some embodiments, the strong acid is selected from the group consisting of: HCl, HI, HBr, HClO₄, HClO₃, HNO₃, H₂SO₄, and a combination thereof.

In some embodiments, the acid is a weak acid. In some embodiments, the weak acid is selected from the group consisting of: phosphorous acid, acetic acid, oxalic acid, phosphoric acid, ascorbic acid, carbonic acid, citric acid, sulfurous acid, tartaric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof. In some embodiments, the weak acid is phosphoric acid.

In some embodiments, the one or more preservatives are present in an amount of about 0.01% to about 2% w/w of the mixture. In some embodiments, the one or more preservatives are present in an amount of about 0.01% to about 1.5%, about 0.01% to about 1.0%, about 0.01% to about 0.5%, about 0.01% to about 0.1%, about 0.01% to about 0.05%, about 1.5% to about 2%, about 1% to about 2%, about 0.5% to about 2%, about 0.1% to about 2%, about 0.05% to about 2%, about 0.01% to about 0.8%, about 0.8% to about 2%, or about 0.1% to about 0.9% w/w of the mixture. For example, about 0.05% to about 0.4%, about 0.05% to about 0.2%, about 0.2% to about 0.5%, about 0.15% to about 0.25%, or about 0.1% to about 0.3% w/w of the mixture. In some embodiments, the one or more preservatives are present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.5%, or about 2% w/w of the mixture.

In some embodiments, the one or more preservatives are selected from the group consisting of: ascorbic acid, an ascorbate, a palmitate, citric acid, a benzoate, benzoic acid, a propionate, propionic acid, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, and a tocopherol. In some embodiments, the one or more preservatives are selected from the group consisting of: a palmitate, a benzoate, benzoic acid, a propionate, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, and a tocopherol. In some embodiments, the ascorbate is selected from the group consisting of: calcium ascorbate, sodium ascorbate, or a combination thereof. In some embodiments, the palmitate is ascorbyl palmitate. In some embodiments, the benzoate is sodium benzoate. In some embodiments, the propionate is selected from the group consisting of: calcium propionate, sodium propionate, and a combination thereof. In some embodiments, the sorbate is selected from the group consisting of: calcium sorbate, potassium sorbate, sodium sorbate, and a combination of two or more thereof. In some embodiments, the erythorbate is sodium erythorbate. In some embodiments, the nitrite is sodium nitrite. In some embodiments, the bisulfite, sulfite, or metabisulfite is selected from the group consisting of: potassium bisulfite, sodium bisulfite, sodium sulfite, sodium metabisulfite, potassium metabisulfite, or combinations thereof.

In some embodiments, the one or more preservatives are sodium benzoate and potassium sorbate. In some embodiments, the sodium benzoate and potassium sorbate are present in an amount of about 0.01% to about 2% w/w of the mixture. In some embodiments, the sodium benzoate and potassium sorbate are present in an amount of about 0.01% to about 1.5%, about 0.01% to about 1.0%, about 0.01% to about 0.5%, about 0.01% to about 0.1%, about 0.01% to about 0.05%, about 1.5% to about 2%, about 1% to about 2%, about 0.5% to about 2%, about 0.1% to about 2%, about 0.05% to about 2%, about 0.01% to about 0.8%, about 0.8% to about 2%, or about 0.1% to about 0.9% w/w of the mixture. For example, about 0.05% to about 0.4%, about 0.05% to about 0.2%, about 0.2% to about 0.5%, about 0.15% to about 0.25%, or about 0.1% to about 0.3% w/w of the mixture. In some embodiments, the sodium benzoate and potassium sorbate are present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.5%, or about 2% w/w of the mixture.

In some embodiments, the sodium benzoate is present in an amount of about 0.01% to about 1.5%, about 0.01% to about 1.0%, about 0.01% to about 0.5%, about 0.01% to about 0.1%, about 0.01% to about 0.05%, about 1.5% to about 2%, about 1% to about 2%, about 0.5% to about 2%, about 0.1% to about 2%, about 0.05% to about 2%, about 0.01% to about 0.8%, about 0.8% to about 2%, or about 0.1% to about 0.9% w/w of the mixture. For example, about 0.05% to about 0.4%, about 0.05% to about 0.2%, about 0.2% to about 0.5%, about 0.15% to about 0.25%, or about 0.1% to about 0.3% w/w of the mixture. In some embodiments, the sodium benzoate is present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.5%, or about 2% w/w of the mixture. In some embodiments, the potassium sorbate is present in an amount of about 0.01% to about 1.5%, about 0.01% to about 1.0%, about 0.01% to about 0.5%, about 0.01% to about 0.1%, about 0.01% to about 0.05%, about 1.5% to about 2%, about 1% to about 2%, about 0.5% to about 2%, about 0.1% to about 2%, about 0.05% to about 2%, about 0.01% to about 0.8%, about 0.8% to about 2%, or about 0.1% to about 0.9% w/w of the mixture. For example, about 0.05% to about 0.4%, about 0.05% to about 0.2%, about 0.2% to about 0.5%, about 0.15% to about 0.25%, or about 0.1% to about 0.3% w/w of the mixture. In some embodiments, the potassium sorbate is present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.5%, or about 2% w/w of the mixture. In some embodiments, the sodium sorbate is present in an amount of 0.05% to about 0.4%, about 0.05% to about 0.2%, about 0.2% to about 0.5%, about 0.15% to about 0.25%, about 0.1% to about 0.3% w/w, or about 0.05% to about 0.15% w/w of the mixture and the sodium benzoate is present in an amount of 0.05% to about 0.4%, about 0.05% to about 0.2%, about 0.2% to about 0.5%, about 0.15% to about 0.25%, about 0.1% to about 0.3%, or about 0.05% to about 0.15% w/w of the mixture. In some embodiments, the sodium sorbate is present in an amount of about 0.01%, about 0.05%, about 0.06%, about 0.08%, about 0.1%, about 0.12%, about 0.14%, about 0.15%, about 0.16%, about 0.18%, about 0.2%, about 0.22% about 0.25%, about 0.26%, about 0.28%, or about 0.3% w/w of the mixture and the sodium benzoate is present in an amount of about 0.01%, about 0.05%, about 0.06%, about 0.08%, about 0.1%, about 0.12%, about 0.14%, about 0.15%, about 0.16%, about 0.18%, about 0.2%, about 0.22% about 0.25%, about 0.26%, about 0.28%, or about 0.3% w/w of the mixture. In some embodiments, the sodium sorbate is present in an amount of about 0.2% w/w of the mixture and the sodium benzoate is present in an amount of about 0.2% w/w of the mixture.

Also provided herein is a method for preparing a partially hydrolyzed fiber comprising:

contacting one or more fibers with an effective amount of an acid to form a mixture, wherein the fiber is present in an amount of about 1% to about 7% w/w of the mixture and the molarity of the acid is about 0.01 to about 0.5 M;

incubating the mixture at about room temperature for a period of time to form a partially hydrolyzed fiber mixture; and

adding one or more preservatives to the partially hydrolyzed fiber mixture, wherein the one or more preservatives are present in an amount of about 0.1% to about 0.5% w/w of the partially hydrolyzed fiber mixture.

Also provided herein is a method for preparing a partially hydrolyzed fiber comprising:

contacting one or more fibers with an effective amount of an acid to form a mixture, wherein the fiber is present in an amount of about 3% to about 5% w/w of the mixture and the molarity of the acid is about 0.01 to about 0.5 M;

incubating the mixture at about room temperature for a period of time to form a partially hydrolyzed fiber mixture; and

adding one or more preservatives to the partially hydrolyzed fiber mixture, wherein the one or more preservatives are present in an amount of about 0.1% to about 0.5% w/w of the partially hydrolyzed fiber mixture.

Also provided herein is a method for preparing a partially hydrolyzed fiber comprising:

contacting one or more fibers with an effective amount of an acid to form a mixture, wherein the fiber is present in an amount of about 4% w/w of the mixture and the molarity of the acid is about 0.013 M;

incubating the mixture at about room temperature for a period of time to form an partially hydrolyzed fiber mixture; and

adding one or more preservatives to the partially hydrolyzed fiber mixture, wherein the one or more preservatives are present in an amount of about 0.2% w/w of the partially hydrolyzed fiber mixture.

Also provided herein is a method for preparing a partially hydrolyzed fiber comprising:

contacting one or more fibers with an effective amount of an acid to form a mixture, wherein the fiber is present in an amount of about 4% to about 8% w/w of the mixture and the molarity of the acid is about 0.01 to about 0.5 M;

incubating the mixture at about room temperature for a period of time to form a partially hydrolyzed fiber mixture; and

adding one or more preservatives to the partially hydrolyzed fiber mixture, wherein the one or more preservatives are present in an amount of about 0.1% to about 0.5% w/w of the partially hydrolyzed fiber mixture.

Also provided herein is a method for preparing a partially hydrolyzed fiber comprising:

contacting one or more fibers with an effective amount of an acid to form a mixture, wherein the fiber is present in an amount of about 5% to about 7% w/w of the mixture and the molarity of the acid is about 0.01 to about 0.5 M;

incubating the mixture at about room temperature for a period of time to form a partially hydrolyzed fiber mixture; and

adding one or more preservatives to the partially hydrolyzed fiber mixture, wherein the one or more preservatives are present in an amount of about 0.1% to about 0.5% w/w of the partially hydrolyzed fiber mixture.

Also provided herein is a method for preparing a partially hydrolyzed fiber comprising:

contacting one or more fibers with an effective amount of an acid to form a mixture, wherein the fiber is present in an amount of about 6% w/w of the mixture and the molarity of the acid is about 0.013 M;

incubating the mixture at about room temperature for a period of time to form an partially hydrolyzed fiber mixture; and

adding one or more preservatives to the partially hydrolyzed fiber mixture, wherein the one or more preservatives are present in an amount of about 0.2% w/w of the partially hydrolyzed fiber mixture.

In some embodiments, the method further comprises adjusting the pH of the partially hydrolyzed fiber mixture to a pH of about 3 to about 5 to form a partially hydrolyzed fiber mixture. In some embodiments, the method further comprises adjusting the pH of the partially hydrolyzed fiber mixture to a pH of about 3.9 to about 4.2.

Also provided herein is a method for preparing a partially hydrolyzed fiber comprising:

contacting one or more fibers with an effective amount of an acid to form a mixture, wherein the fiber is present in an amount of about 0.5% to about 5% w/w of the mixture, the molarity of the acid is about 0.01 to about 0.1 M, and the pH of the mixture is about 1.5 to about 2.5;

heating the mixture to about 60° C. to about 75° C. to form a heated mixture;

holding the heated mixture at about 50° C. to about 80° C. for a period of time to form a heated and incubated mixture;

cooling the heated and incubated mixture to form a partially hydrolyzed fiber mixture; and

adding one or more preservatives to the partially hydrolyzed fiber, wherein the one or more preservatives are present in an amount of about 0.1% to about 0.5% w/w of the partially hydrolyzed fiber mixture.

Also provided herein is a method for preparing a partially hydrolyzed fiber comprising:

contacting one or more fibers with an effective amount of an acid to form a mixture, wherein the fiber is present in an amount of about 0.5% to about 3% w/w of the mixture, the molarity of the acid is about 0.01 to about 0.08 M, and the pH of the mixture is about 1.5 to about 2.5;

heating the mixture to about 60° C. to about 75° C. to form a heated mixture;

holding the heated mixture at about 50° C. to about 80° C. for a period of time to form a heated and incubated mixture;

cooling the heated and incubated mixture to form a partially hydrolyzed fiber mixture; and

adding one or more preservatives to the partially hydrolyzed fiber mixture, wherein the one or more preservatives are present in an amount of about 0.1% to about 0.3% w/w of the partially hydrolyzed fiber mixture.

Also provided herein is a method for preparing a partially hydrolyzed fiber comprising:

contacting one or more fibers with an effective amount of an acid to form a mixture, wherein the fiber is present in an amount of about 2% w/w of the mixture, the molarity of the acid is about 0.5 M, and the pH of the mixture is about 1.8;

heating the mixture to about 60° C. to about 75° C. to form a heated mixture;

holding the heated mixture at about 50° C. to about 80° C. for a period of time to form a heated and incubated mixture;

cooling the heated and incubated mixture to form a partially hydrolyzed fiber mixture; and

adding one or more preservatives to the partially hydrolyzed fiber mixture, wherein the one or more preservatives are present in an amount of about 0.2% w/w of the partially hydrolyzed fiber mixture.

In some embodiments, the mixture is heated to about 65° C. to about 70° C. In some embodiments, the heated mixture is held at a temperature of about 65° C. to about 70° C. In some embodiments, the mixture is heated rapidly.

In some embodiments, the method further comprises stirring the mixture during heating. In some embodiments, the method further comprises stirring the heated mixture during the period of time.

In some embodiments, water is added to the heated and incubated mixture to return to the original, pre-heated weight. In some embodiments, the heated and incubated mixture is cooled rapidly. In some embodiments, the method further comprises stirring the heated and incubated mixture during cooling. In some embodiments, the heated and incubated mixture is cooled to about room temperature.

In some embodiments, the method further comprises filtering the partially hydrolyzed fiber mixture.

In some embodiments, the method further comprises adjusting the pH of the partially hydrolyzed fiber mixture to a pH of about 3 to about 5. In some embodiments, the method further comprises adjusting the pH of the partially hydrolyzed fiber mixture to a pH of about 3.5 to about 4. In some embodiments, the method further comprises adjusting the pH of the partially hydrolyzed fiber mixture to a pH of about 3.8.

In some embodiments, the fiber is selected from the group consisting of: psyllium, pectin, guar, a lignin, cellulose, hemicellulose, resistant starch, a resistant dextrin, inulin, a chitin, pectin, a beta-glucan, an oligosaccharide, potato starch, and a combination of two or more thereof. In some embodiments, the fiber is psyllium.

In some embodiments, the acid is selected from the group consisting of: HCl, HI, HBr HClO₄, HClO₃, HNO₃, H₂SO₄, phosphoric acid, phosphorous acid, acetic acid, oxalic acid, ascorbic acid, carbonic acid, sulfurous acid, tartaric acid, citric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof. In some embodiments, the acid is phosphoric acid. In some embodiments, the acid is citric acid.

In some embodiments, the acid is a strong acid. In some embodiments, the strong acid is selected from the group consisting of: HCl, HI, HBr, HClO₄, HClO₃, HNO₃, H₂SO₄, and a combination thereof.

In some embodiments, the acid is a weak acid In some embodiments, the weak acid is selected from the group consisting of: phosphorous acid, acetic acid, oxalic acid, phosphoric acid, ascorbic acid, carbonic acid, citric acid, sulfurous acid, tartaric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof. In some embodiments, the weak acid is phosphoric acid.

In some embodiments, the one or more preservatives are selected from the group consisting of: a palmitate, a benzoate, benzoic acid, a propionate, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, and a tocopherol. In some embodiments, the one or more preservatives are sodium benzoate and potassium sorbate. In some embodiments, the sodium benzoate is present in an amount of about 0.1% w/w of the partially hydrolyzed fiber mixture and the potassium sorbate is present in an amount of about 0.1% w/w of the partially hydrolyzed fiber mixture.

In some embodiments, the method further comprises adding one or more sweeteners to the partially hydrolyzed fiber mixture.

In some embodiments, the method further comprises adding one or more flavoring agents to the partially hydrolyzed fiber mixture.

### Methods of Use

The compositions provided herein can be used in a variety of methods, including both the treatment of a disease, disorder, or condition, or to improve a disease, disorder, or condition, for example, one or more symptoms associated with the disease, disorder, or condition.

In some embodiments, provided herein are methods of treating a gastrointestinal disorder in a subject in need thereof comprising administering to the subject a composition or dietary fiber combination as described herein. In some embodiments, provided herein are methods of improving a gastrointestinal disorder in a subject in need thereof comprising administering to the subject a composition or dietary fiber combination as described herein. In some embodiments, provided herein are methods for improving or maintaining intestinal health in a subject in need thereof comprising administering to the subject a composition or dietary fiber combination as described herein. Also provided herein are methods for supporting a healthy digestive system in a subject in need thereof comprising administering to the subject a composition or dietary fiber combination as described herein. In some embodiments, provided herein are methods for altering the gut microbiome in a favorable manner in a subject in need thereof comprising administering to the subject a composition or dietary fiber combination as described herein. In some embodiments, a method for altering the gut microbiome in a favorable manner comprises decreasing the amount of bacteria that increase gut inflammation and/or decrease SCFA production. In some embodiments, a method for altering the gut microbiome in a favorable manner comprises increasing the amount of bacteria that decrease gut inflammation and/or increase SCFA production. In some embodiments, the gut microbiome may be analyzed by 16S RRNA unit subtyping. Also provided herein are methods for reducing inflammation in a subject in need thereof comprising administering to the subject a composition or dietary fiber combination as described herein. In some embodiments, the subject has a gastrointestinal disorder.

In some embodiments, assessing a gastrointestinal disorder and/or gut microbiome health may comprise determining erythrocyte sedimentation rate (ESR), measuring the amount of C-reactive protein (CRP) (e.g., conducting a CRP test), or performing a clinical assessment to assess the severity or improvement of symptoms such as abdominal pain, bloating, constipation, diarrhea, nausea, dyspepsia. In some embodiments, a symptom score for diarrhea or constipation can be measured. A clinical assessment and/or symptom score may include, for example, the Bristol stool scale and Rome criteria (see, e.g., Lacy and Patel. J. Clin. Med. 2017; 6(11): 99, which is incorporated by reference herein in its entirety).

In some embodiments, treating a gastrointestinal disorder in a subject comprises reducing one or more symptoms in the subject. For example, reducing flatulence, intestinal gas, abdominal pain, bloating, constipation, diarrhea, nausea, and/or dyspepsia. In some embodiments, the method includes administering to the subject any of the compositions as described herein. In some embodiments, the method includes administering a therapeutically effective amount of any of the compositions as described herein to the subject.

In some embodiments, improving a gastrointestinal disorder in a subject comprises reducing one or more symptoms in the subject. For example, reducing flatulence, intestinal gas, abdominal pain, bloating, constipation, diarrhea, nausea, and/or dyspepsia. In some embodiments, the method includes administering a therapeutically effective amount of any of the compositions as described herein to the subject. In some embodiments, improving a gastrointestinal disorder in a subject comprises providing relief from one or more symptoms in the subject selected from the group consisting of: flatulence, intestinal gas, abdominal pain, bloating, constipation, diarrhea, nausea, and/or dyspepsia. In some embodiments, the method includes administering to the subject any of the compositions as described herein.

In some embodiments, improving a state of diarrhea in a subject can refer to, for example, decreasing bowel movements, decreasing sudden urgency to defecate, and/or increasing stool hardness. In some embodiments, improving a state of constipation can refer to, for example, increasing bowel movements and/or softening of the stool.

In some embodiments, the gastrointestinal disorder is selected from the group consisting of: a foregut disease, flatulence, intestinal gas, bloating, leaky gut syndrome, constipation, hemorrhoid, anal fissure, diarrhea, fecal incontinence, irritable bowel syndrome, inflammatory bowel disease (Crohn's disease and ulcerative colitis), microscopic colitis, small intestinal bacterial overgrowth, certain metabolic syndromes such as glucose intolerance, prediabetes, diabetes, coronary artery disease, nonalcoholic fatty liver disease, steatohepatitis, and obesity. In some embodiments, the gastrointestinal disorder (e.g., colitis) is induced by one or more chemotherapeutic agents, by treatment with adoptive cell therapy (e.g., immunotherapy), or by one or more alloimmune diseases (such as acute or chronic graft-vs-host disease). Non-limiting examples of a foregut disease include GERD, Barrett's esophagus, esophageal cancer (e.g., squamous or adenocarcinoma esophageal cancer), esophageal motility disorders, eosinophilic esophagitis, achalasia, oral cancer, halitosis, achalasia, esophageal diverticula, and periodontal gum disease.

In some embodiments, improving a gastrointestinal disorder in a subject in need thereof includes improving (e.g., decreasing) blood glucose levels in the subject. For example, the blood glucose level in the subject can decrease by at least about 10 mg/dl, e.g., at least 20 mg/dl, 30 mg/dl, 40 mg/dl, 50 mg/dl, 60 mg/dl, 70 mg/dl, 80 mg/dl, 90 mg/dl, 100 mg/dl or more. In some embodiments, the gastrointestinal disorder is diabetes or prediabetes. In some embodiments, the subject is administered about 0.5% to about 10% w/w of the composition of a first fiber; and about 0.5% to about 10% w/w of the composition of a second fiber. In some embodiments, the subject is administered about 2% to about 6% w/w of the composition of a first fiber; and about 2% to about 6% w/w of the composition of a second fiber. In some embodiments, the subject is administered about 4% w/w of the composition of the first fiber. In some embodiments, the subject is administered about 4% w/w of the composition of the second fiber. In some embodiments, the subject is administered about 3% w/w of the composition of the first fiber. In some embodiments, the subject is administered about 3% w/w of the composition of the second fiber.

In some embodiments, the subject is administered about 1 gram to about 6 grams of the first fiber; and about 1 gram to about 6 grams of the second fiber. In some embodiments, the subject is administered about 1 grams to about 4 grams of the first fiber; and about 1 grams to about 4 grams of the second fiber. In some embodiments, the subject is administered about 2 grams to about 3 grams of the first fiber; and about 2 grams to about 3 grams of the second fiber. In some embodiments, the subject is administered about 1.8 grams of the first fiber and about 1.8 grams of the second fiber. In some embodiments, the subject is administered about 2.4 grams of the first fiber and about 2.4 grams of the second fiber. In some embodiments, the subject is administered about 2.5 grams of the first fiber and about 2.5 grams of the second fiber. In some embodiments, the subject is administered about 4 grams of the first fiber and about 4 grams of the second fiber. In some embodiments, the subject is administered about 1 gram to about 6 grams of the first fiber. In some embodiments, the subject is administered about 1 gram to about 6 grams of the second fiber. In some embodiments, the subject is administered about 1 grams to about 4 grams of the first fiber. In some embodiments, the subject is administered about 1 grams to about 4 grams of the second fiber. In some embodiments, the subject is administered about 3 grams to about 5 grams of the first fiber. In some embodiments, the subject is administered about 3 grams to about 5 grams of the second fiber. In some embodiments, the subject is administered about 2 grams to about 3 grams of the first fiber. In some embodiments, the subject is administered about 2 grams to about 3 grams of the second fiber. In some embodiments, the subject is administered about 1 gram, about 1.8 grams, about 2 grams, about 2.4 grams, about 2.5 grams, about 3 grams, or about 4 grams of the first fiber. In some embodiments, the composition about 1 gram, about 1.8 grams, about 2 grams, about 2.4 grams, about 2.5 grams, about 3 grams, or about 4 grams of the second fiber.

In some embodiments, the subject is administered a first fiber and a second fiber at a ratio of about 1:4 to about 4:1 first fiber:second fiber. For example, about 1:3, about 2:5, about 1:2, about 2:3, about 1:1, about 2:1, about 5:2, about 3:1, or about 3:2 first fiber:second fiber.

In some embodiments, the first fiber is wheat dextrin and the second fiber is inulin. In some embodiments, the first fiber is pectin and the second fiber is inulin. In some embodiments, the first fiber is pectin and the second fiber is potato starch. In some embodiments, the first fiber is potato starch and the second fiber is inulin. In some embodiments, the first fiber is wheat dextrin and the second fiber is potato starch. In some embodiments, the first fiber is wheat dextrin and the second fiber is pectin.

For example, in some embodiments, a subject is administered a first fiber and then subsequently or simultaneously is administered a second fiber. In some embodiments, the first fiber is present as a first composition and the second fiber is present as a second composition.

In some embodiments, a method as described herein comprises administering to the subject:

a first composition; and

a second composition.

In some embodiments, the first composition comprises about 2% to about 6% w/w of the composition of the first fiber. In some embodiments, the first composition comprises about 3% to about 5% w/w of the composition of the first fiber. In some embodiments, the first composition comprises about 4% w/w of the composition of the first fiber. In some embodiments, the first composition comprises about 3% w/w of the composition of the first fiber. In some embodiments, the first fiber is wheat dextrin, potato starch, pectin, or a combination thereof.

In some embodiments, the second composition comprises about 2% to about 6% w/w of the composition of the second fiber. In some embodiments, the second composition comprises about 3% to about 5% w/w of the composition of the second fiber. In some embodiments, the second composition comprises about 4% w/w of the composition of the second fiber. In some embodiments, the second composition comprises about 3% w/w of the composition of the second fiber. In some embodiments, the second fiber is inulin, potato starch, pectin, or a combination thereof.

In some embodiments, the first fiber in the first composition and the second fiber in the second composition are different.

In some embodiments, the ratio of the first fiber in the first composition to the second fiber in the second composition is about 1:4 to about 4:1 first fiber:second fiber. For example, about 1:3, about 2:5, about 1:2, about 2:3, about 1:1, about 2:1, about 5:2, about 3:1, or about 3:2 first fiber:second fiber.

In some embodiments, a method as described herein comprises administering to the subject:

a first composition comprising about 2% to about 6% w/w of the composition of wheat dextrin; and

a second composition comprising about 2% to about 6% w/w of the composition of inulin, potato starch, pectin, or a combination thereof. In some embodiments, the first composition comprises about 3% w/w of the composition of wheat dextrin. In some embodiments, the first composition comprises about 4% w/w of the composition of wheat dextrin. In some embodiments, the second composition comprises about 3% w/w of the composition of inulin, potato starch, pectin, or a combination thereof. In some embodiments, the second composition comprises about 4% w/w of the composition of inulin, potato starch, pectin, or a combination thereof.

In some embodiments, a method as described herein comprises administering to the subject:

a first composition comprising about 3% to about 5% w/w of the composition of wheat dextrin; and

a second composition comprising about 3% to about 5% w/w of the composition of inulin, potato starch, pectin, or a combination thereof.

In some embodiments, a method as described herein comprises administering to the subject:

a first composition comprising about 4% w/w of the composition of wheat dextrin; and

a second composition comprising about 4% w/w of the composition of inulin, potato starch, pectin, or a combination thereof.

In some embodiments, a method as described herein comprises administering to the subject:

a first composition comprising about 4% w/w of the composition of wheat dextrin; and

a second composition comprising about 3% w/w of the composition of inulin, potato starch, pectin, or a combination thereof.

In some embodiments, a method as described herein comprises administering to the subject:

a first composition comprising about 3% w/w of the composition of wheat dextrin; and

a second composition comprising about 3% w/w of the composition of inulin, potato starch, pectin, or a combination thereof.

In some embodiments, a method as described herein comprises administering to the subject:

a first composition comprising about 2% to about 6% w/w of the composition of pectin; and

a second composition comprising about 2% to about 6% w/w of the composition of inulin, potato starch, or a combination thereof. In some embodiments, the first composition comprises about 3% w/w of the composition of pectin. In some embodiments, the first composition comprises about 4% w/w of the composition of pectin. In some embodiments, the second composition comprises about 3% w/w of the composition of inulin, potato starch, or a combination thereof. In some embodiments, the second composition comprises about 4% w/w of the composition of inulin, potato starch, or a combination thereof.

In some embodiments, a method as described herein comprises administering to the subject:

a first composition comprising about 3% to about 5% w/w of the composition of pectin; and

a second composition comprising about 3% to about 5% w/w of the composition of inulin, potato starch, or a combination thereof.

In some embodiments, a method as described herein comprises administering to the subject:

a first composition comprising about 4% w/w of the composition of pectin; and

a second composition comprising about 4% w/w of the composition of inulin, potato starch, or a combination thereof.

In some embodiments, a method as described herein comprises administering to the subject:

a first composition comprising about 3% w/w of the composition of pectin; and

a second composition comprising about 4% w/w of the composition of inulin, potato starch, or a combination thereof.

In some embodiments, a method as described herein comprises administering to the subject:

a first composition comprising about 3% w/w of the composition of pectin; and

a second composition comprising about 3% w/w of the composition of inulin, potato starch, or a combination thereof.

In some embodiments, a method as described herein comprises administering to the subject:

a first composition comprising about 2% to about 6% w/w of the composition of potato starch; and

a second composition comprising about 2% to about 6% w/w of the composition of inulin. In some embodiments, the first composition comprises about 3% w/w of the composition of potato starch. In some embodiments, the first composition comprises about 4% w/w of the composition of potato starch. In some embodiments, the second composition comprises about 3% w/w of the composition of inulin. In some embodiments, the second composition comprises about 4% w/w of the composition of inulin.

In some embodiments, a method as described herein comprises administering to the subject:

a first composition comprising about 3% to about 5% w/w of the composition of potato starch; and

a second composition comprising about 3% to about 5% w/w of the composition of inulin.

In some embodiments, a method as described herein comprises administering to the subject:

a first composition comprising about 4% w/w of the composition of potato starch; and

a second composition comprising about 4% w/w of the composition of inulin.

In some embodiments, a method as described herein comprises administering to the subject:

a first composition comprising about 3% w/w of the composition of potato starch; and

a second composition comprising about 4% w/w of the composition of inulin.

In some embodiments, a method as described herein comprises administering to the subject:

a first composition comprising about 3% w/w of the composition of potato starch; and

a second composition comprising about 3% w/w of the composition of inulin.

In some embodiments, the first composition and the second composition are administered concurrently. In some embodiments, the first composition and the second composition are mixed prior to administration. In some embodiments, the first composition is administered before the second composition. In some embodiments, the second composition is administered before the first composition.

In some embodiments, the first composition and the second composition are administered daily.

In some embodiments, a method as described herein comprises administering to the subject a composition comprising:

a first fiber; and

a second fiber.

In some embodiments, the composition comprises about 1 grams to about 6 grams of the first fiber; and about 1 grams to about 6 grams of the second fiber. In some embodiments, the composition comprises about 4 grams of the first fiber; and about 4 grams of the second fiber. In some embodiments, the composition comprises about 1.8 grams of the first fiber; and about 1.8 grams of the second fiber. In some embodiments, the composition comprises about 2.4 grams of the first fiber; and about 2.4 grams of the second fiber. In some embodiments, the composition comprises about 1.8 grams of the first fiber; and about 2.4 grams of the second fiber. In some embodiments, the composition comprises about 2.4 grams of the first fiber; and about 1.8 grams of the second fiber. In some embodiments, the composition comprises about 2.5 grams of the first fiber; and about 2.5 grams of the second fiber.

In some embodiments, the ratio of the first fiber in the first composition to the second fiber in the second composition is about 1:4 to about 4:1 first fiber:second fiber. For example, about 1:3, about 2:5, about 1:2, about 2:3, about 1:1, about 2:1, about 5:2, about 3:1, or about 3:2 first fiber:second fiber.

In some embodiments, the first fiber is wheat dextrin and the second fiber is inulin. In some embodiments, the first fiber is pectin and the second fiber is inulin. In some embodiments, the first fiber is pectin and the second fiber is potato starch. In some embodiments, the first fiber is potato starch and the second fiber is inulin. In some embodiments, the first fiber is wheat dextrin and the second fiber is potato starch. In some embodiments, the first fiber is wheat dextrin and the second fiber is pectin.

In some embodiments, a method as described herein comprises administering to the subject a composition comprising:

pectin or wheat dextrin present in amount of about 0.5% to about 10% w/w of the composition;

inulin, potato starch, or a combination thereof, present in amount of about 0.5% to about 10% w/w of the composition.

In some embodiments, a method as described herein comprises administering to the subject a composition comprising:

pectin or wheat dextrin present in an amount of about 3% to about 5% w/w of the composition; and

inulin, potato starch, or a combination thereof, present in an amount of about 3% to about 5% w/w of the composition.

In some embodiments, a method as described herein comprises administering to the subject a composition comprising:

pectin or wheat dextrin present in an amount of about 2% to about 6% w/w of the composition; and

inulin, potato starch, or a combination thereof, present in an amount of about 2% to about 6% w/w of the composition.

In some embodiments, a method as described herein comprises administering to the subject a composition comprising:

pectin or wheat dextrin present in an amount of about 4% w/w of the composition; and

inulin, potato starch, or a combination thereof, present in an amount of about 4% w/w of the composition.

In some embodiments, a method as described herein comprises administering to the subject a composition comprising:

pectin or wheat dextrin present in an amount of about 3% w/w of the composition; and

inulin, potato starch, or a combination thereof, present in an amount of about 4% w/w of the composition.

In some embodiments, a method as described herein comprises administering to the subject a composition comprising:

pectin or wheat dextrin present in an amount of about 4% w/w of the composition; and

inulin, potato starch, or a combination thereof, present in an amount of about 3% w/w of the composition.

In some embodiments, a method as described herein comprises administering to the subject a composition comprising:

pectin or wheat dextrin present in an amount of about 3% w/w of the composition; and

inulin, potato starch, or a combination thereof, present in an amount of about 3% w/w of the composition.

In some embodiments, the composition comprises about 1 gram to about 4 grams of pectin or wheat dextrin; and about 1 gram to about 4 grams of inulin, potato starch, or a combination thereof. In some embodiments, the composition comprises about 2 grams to about 3 grams of pectin or wheat dextrin; and about 2 grams to about 3 grams of inulin, potato starch, or a combination thereof. In some embodiments, the composition comprises about 4 grams of pectin or wheat dextrin; and about 4 grams of inulin, potato starch, or a combination thereof. In some embodiments, the composition comprises about 3 grams of pectin or wheat dextrin; and about 3 grams of inulin, potato starch, or a combination thereof. In some embodiments, the composition comprises about 2.5 grams of pectin or wheat dextrin; and about 2.5 grams of inulin, potato starch, or a combination thereof. In some embodiments, the composition comprises about 2.4 grams of pectin or wheat dextrin; and about 2.4 grams of inulin, potato starch, or a combination thereof. In some embodiments, the composition comprises about 1.8 grams of pectin or wheat dextrin; and about 1.8 grams of inulin, potato starch, or a combination thereof.

In some embodiments, a method as described herein comprises administering to the subject a composition comprising:

wheat dextrin or potato starch present in amount of about 0.5% to about 10% w/w of the composition;

inulin, pectin, or a combination thereof, present in amount of about 0.5% to about 10% w/w of the composition.

In some embodiments, a method as described herein comprises administering to the subject a composition comprising:

wheat dextrin or potato starch present in an amount of about 3% to about 5% w/w of the composition; and

inulin, pectin, or a combination thereof, present in an amount of about 3% to about 5% w/w of the composition.

In some embodiments, a method as described herein comprises administering to the subject a composition comprising:

wheat dextrin or potato starch present in an amount of about 2% to about 6% w/w of the composition; and

inulin, pectin, or a combination thereof, present in an amount of about 2% to about 6% w/w of the composition.

In some embodiments, a method as described herein comprises administering to the subject a composition comprising:

wheat dextrin or potato starch present in an amount of about 4% w/w of the composition; and

inulin, pectin, or a combination thereof, present in an amount of about 4% w/w of the composition.

In some embodiments, a method as described herein comprises administering to the subject a composition comprising:

wheat dextrin or potato starch present in an amount of about 3% w/w of the composition; and

inulin, pectin, or a combination thereof, present in an amount of about 4% w/w of the composition.

In some embodiments, a method as described herein comprises administering to the subject a composition comprising:

wheat dextrin or potato starch present in an amount of about 4% w/w of the composition; and

inulin, pectin, or a combination thereof, present in an amount of about 3% w/w of the composition.

In some embodiments, a method as described herein comprises administering to the subject a composition comprising:

wheat dextrin or potato starch present in an amount of about 3% w/w of the composition; and

inulin, pectin, or a combination thereof, present in an amount of about 3% w/w of the composition.

In some embodiments, the composition comprises about 1 gram to about 4 grams of wheat dextrin or potato starch; and about 1 gram to about 4 grams of inulin, pectin, or a combination thereof. In some embodiments, the composition comprises about 2 grams to about 3 grams of wheat dextrin or potato starch; and about 2 grams to about 3 grams of inulin, pectin, or a combination thereof. In some embodiments, the composition comprises about 4 grams of wheat dextrin or potato starch; and about 4 grams of inulin, pectin, or a combination thereof. In some embodiments, the composition comprises about 3 grams of wheat dextrin or potato starch; and about 3 grams of inulin, pectin, or a combination thereof. In some embodiments, the composition comprises about 2.5 grams of wheat dextrin or potato starch; and about 2.5 grams of inulin, pectin, or a combination thereof. In some embodiments, the composition comprises about 2.4 grams of wheat dextrin or potato starch; and about 2.4 grams of inulin, pectin, or a combination thereof. In some embodiments, the composition comprises about 1.8 grams of wheat dextrin or potato starch; and about 1.8 grams of inulin, pectin, or a combination thereof.

In some embodiments, the composition administered to a subject as described herein comprises one or more preservatives present in an amount of about 0.01% to about 2% w/w of the composition. In some embodiments, the pH of the composition is configured to be about 3.9 to about 4.2.

In some embodiments, a method as described herein comprises administering to the subject a composition comprising:

a partially hydrolyzed psyllium present in amount of about 0.5% to about 10% w/w of the composition;

phosphoric acid; and

one or more preservatives present in an amount of about 0.01% to about 2% w/w of the composition.

In some embodiments, the molarity of the phosphoric acid is about 0.03 M to about 0.07 M. In some embodiments, the pH of the composition is about 3.7 to about 3.9. In some embodiments, the partially hydrolyzed psyllium is present in amount of about 2% w/w of the composition; and the sodium sorbate and sodium benzoate together are present in an amount of about 0.2% w/w of the composition. In some embodiments, the molarity of the phosphoric acid is 0.05 M. In some embodiments, the pH of the composition is about 3.8.

In some embodiments, a method as described herein comprises administering to the subject a composition comprising:

a partially hydrolyzed pectin present in amount of about 0.5% to about 10% w/w of the composition;

citric acid; and

one or more preservatives present in an amount of about 0.01% to about 2% w/w of the composition. In some embodiments, the molarity of the citric acid is about 0.01 M to about 0.015 M.

In some embodiments, the pH of the composition is about 3.9 to about 4.2. In some embodiments, the partially hydrolyzed pectin is present in amount of about 4% w/w of the composition; and the sodium sorbate and sodium benzoate together are present in an amount of about 0.2% w/w of the composition. In some embodiments, the partially hydrolyzed pectin is present in amount of about 6% w/w of the composition; and the sodium sorbate and sodium benzoate together are present in an amount of about 0.2% w/w of the composition. In some embodiments, the molarity of the acid is 0.013 M. In some embodiments, the pH of the composition is about 3.9 to about 4.2.

In some embodiments, the subject is a child, an adolescent, or an elderly subject.

In some embodiments, the subject is administered about 1 gram to about 15 grams of a fiber as described herein. For example, the total amount of a first fiber, a second fiber, a third fiber, etc. administered to a subject can be about 1 gram to about 15 grams. In some embodiments, a subject is administered about 1 gram to about 2 grams, 1 gram to about 3 grams, 1 gram to about 4 grams, 1 gram to about 5 grams, 1 gram to about 6 grams, 1 gram to about 7 grams, 1 gram to about 8 grams, 1 gram to about 9 grams, 1 gram to about 10 grams, 1 gram to about 11 grams, 1 gram to about 12 grams, 1 gram to about 13 grams, 1 gram to about 14 grams, about 14 grams to about 15 grams, about 13 grams to about 15 grams, about 12 grams to about 15 grams, about 11 grams to about 15 grams, about 10 grams to about 15 grams, about 9 grams to about 15 grams, about 8 grams to about 15 grams, about 7 grams to about 15 grams, about 6 grams to about 15 grams, about 5 grams to about 15 grams, about 4 grams to about 15 grams, about 3 grams to about 15 grams, or about 2 grams to about 15 grams of a fiber as described herein. For example, about 3 grams to about 6 grams, about 4 grams to about 7 grams, about 5 grams to about 8 grams, or about 4 to about 8 grams of a fiber as described herein. In some embodiments, a subject is administered about 1 gram, about 2 grams, about 2.5 grams, about 3 grams, about 3.5 grams, about 4 grams, about 4.5 grams, about 5 grams, about 5.5 grams, about 6 grams, about 6.5 grams, about 7 grams, about 7.5 grams, about 8 grams, about 8.5 grams, about 9 grams, about 10 grams, about 11 grams, about 12 grams, about 13 grams, about 14 grams, or about 15 grams.

In some embodiments, the remaining part of a composition used in a method described herein is water or buffered water. In some embodiments, the composition is a liquid composition. In some embodiments, the liquid composition is selected from the group consisting of: a solution, a syrup, a suspension, and a concentrate.

### EXAMPLES

The invention will be described in greater detail by way of specific examples. The following examples are offered for illustrative purposes, and to exemplify the oral compositions and methods described herein and are not intended to limit the invention in any manner. Many variations will suggest themselves and are within the full-intended scope. Those of skill in the art will readily recognize a variety of non-critical parameters that can be changed or modified to yield essentially the same results.

### Example 1. Acid Hydrolysis and Partially Hydrolyzed Psyllium Drink Preparation

### Materials

TABLE 1
Ingredients for acid hydrolysis of psyllium

| | % w/w of the |
|---|---|
| Ingredients | composition |
| Aqueous H₃PO₄ Solution | 97.8 |
| Psyllium Husk Powder - 95% (60 mesh) | 2 |
| Potassium Sorbate | 0.1 |
| Sodium Benzoate | 0.1 |
| NaOH or H₃PO₄ to pH ~3.8 | Q.S. |

### Preparation

The psyllium husk powder was added to the H₃PO₄ solution (0.05 M H₃PO₄, pH 1.89). The mixture was heated rapidly to about 65-70° C. with propellar mixing. The temperature was kept at 65-70° C. for about 6.5 hours (e.g., the minimum amount of time for the mixture to be water thin at room temperature). Distilled water or ice was added to return to the original pre-heating weight/volume. The mixture was removed from heat and cooled rapidly to room temperature with mixing. The sodium benzoate and potassium sorbate were added and mixed until dissolved. The mixture was filtered and the pH was brought to about 3.7 to 3.9.

TABLE 2
Comparison of partially hydrolyzed psyllium preparations

| | | | | | pH | | |
|---|---|---|---|---|---|---|---|
| | | | | | (After | | pH |
| | | pH of | | | Heat | | (After |
| Prepa- | H₃PO₄ | H₃PO₄ | Temp | Time | & | | NaOH |
| ration | Molarity | Soln | (° C.) | (hrs) | Dilution) | Viscosity | Add'n) |
| 1 | 0.030 | 2.04 | 65-70 | 3.0 | 2.7 | Too | — |
| | | | | | | Thick | |
| 2 | 0.050 | 1.89 | 65-70 | 6.5 | 2.22 | Good | 3.00 |

### Results

Preparation #1 resulted in an unacceptable gel, whereas Preparation #2 resulted in a solution that was slightly thicker than water, but filtered well. Preparation #2 was also slightly cloudy, slightly yellowish, and was an acceptable gel.

### Example 2. Acid Hydrolysis and Partially Hydrolyzed Pectin Drink Preparation

### Materials

TABLE 2
Ingredients for acid hydrolysis of pectin

| | % w/w of the |
|---|---|
| Ingredients | composition |
| Water | 95.55 |
| Potassium Sorbate | 0.1 |
| Sodium Benzoate | 0.1 |
| Anhydrous (SOLID) Citric Acid | 0.25 |
| Sodium Hydroxide (18% Solution) | Q.S. |
| Pectin Powder | 4 |
| pH Adjusted to pH = 3.9-4.2 | Q.S. |

### Preparation

The water was added to a beaker followed by the potassium sorbate and sodium benzoate. The solution was mixed until the potassium sorbate and sodium benzoate were dissolved. The citric acid was then added to the beaker with mixing. The pectin powder was added at room temperature with vigorous mixing. The pH and viscosity were monitored during mixing. When the desired viscosity or pH was achieved, the pH of the mixture was adjusted to about 3.9 to about 4.2.

### Other Embodiments

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

1. A composition comprising:
an oligosaccharide present in an amount of about 5% to about 9% w/w of the composition; and
a fiber present in an amount of about 5% to about 9% w/w of the composition, wherein the fiber is selected from the group consisting of: pectin, inulin, psyllium, guar, a lignin, cellulose, hemicellulose, resistant starch, a resistant dextrin, a chitin, a beta-glucan, potato starch, and a combination thereof.

2. The composition of claim 1, wherein the oligosaccharide is present in an amount of about 6% to about 7% w/w of the composition.

3. The composition of claim 1, wherein the oligosaccharide is present in an amount of about 6.5% w/w of the composition.

4. The composition of claim 1, wherein the oligosaccharide is selected from the group consisting of: fructooligosaccharide, maltose, iso-maltose, a maltosyl-iso-malto-oligosaccharide, and a combination thereof.

5. The composition of claim 4, wherein the oligosaccharide is maltose.

6. The composition of claim 1, wherein the fiber is present in an amount of about 6% to about 7% w/w of the composition.

7. The composition of claim 1, wherein the fiber is present in an amount of about 6.5% w/w of the composition.

8. The composition of claim 1, wherein the fiber is selected from the group consisting of: pectin, inulin, wheat dextrin, potato starch, and a combination thereof.

9. The composition of claim 8, wherein the fiber is pectin.

10. The composition of claim 8, wherein the fiber is inulin.

11. The composition of claim 8, wherein the fiber is wheat dextrin.

12. The composition of claim 8, wherein the fiber is potato starch.

13.-29. (canceled)

30. The composition of claim 1, wherein the composition further comprises one or more of: an acid, a preservative, and a flavoring agent.

31.-51. (canceled)

52. The composition of claim 1, wherein the composition is a gel composition.

53. A method for reducing inflammation in a subject in need thereof comprising administering to the subject a composition of claim 1.

54. A method for improving intestinal health in a subject in need thereof comprising administering to the subject a composition of claim 1.

55. A method for improving a gastrointestinal disease in a subject in need thereof comprising administering to the subject a composition of claim 1.

56. The method of claim 55, wherein the gastrointestinal disease is selected from gastrointestinal disorder is selected from the group consisting of: a foregut disease, flatulence, intestinal gas, bloating, leaky gut syndrome, constipation, hemorrhoid, anal fissure, diarrhea, fecal incontinence, irritable bowel syndrome, inflammatory bowel disease, microscopic colitis, small intestinal bacterial overgrowth, glucose intolerance, prediabetes, diabetes, coronary artery disease, nonalcoholic fatty liver disease, steatohepatitis, and obesity.

57. The method of claim 56, wherein the forgut disease is selected from the group consisting of: GERD, Barrett's esophagus, esophageal cancer, esophageal motility disorders, eosinophilic esophagitis, achalasia, oral cancer, halitosis, achalasia, esophageal diverticula, and periodontal gum disease.
